(19) United States
(12) Patent Application Publication
(10) Pub. No.: US 2022/0341944 A1
(43) Pub. Date: Oct. 27, 2022

(54) BIOMARKER COMBINATIONS IN EX VIVO LUNG PERFUSION (EVLP) PERFUSATE

(71) Applicant: UNIVERSITY HEALTH NETWORK, Toronto (CA)

(72) Inventors: Shafique Keshavjee, Toronto (CA); Mingyao Liu, North York (CA); Marcelo Cypel, Toronto (CA); Andrew Sage, Toronto (CA)

(21) Appl. No.: 17/413,772

(22) PCT Filed: Dec. 13, 2019
(86) PCT No.: PCT/CA2019/051813

§ 371 Date: Jun. 14, 2021

Related U.S. Application Data
(60) Provisional application No. 62/779,220, filed on Dec. 13, 2018.
(60) Provisional application No. 62/796,942, filed on Jan. 25, 2019.

(51) Int. Cl. G01N 33/68; G16B 40/20; G16H 10/40; G16H 50/30

(52) U.S. Cl. CPC G01N 33/6884; G16B 40/20; G16H 10/40; G16H 50/30; G01N 2333/54; G01N 2333/70578; G01N 2800/50

(57) ABSTRACT

Methods and kits for screening, diagnosing, detecting or predicting a patient outcome/risk variable for a lung transplant recipient after transplant or an EVLP outcome by measuring biomarker levels of at least three biomarkers selected from IL-6, IL-8, IL-10 and IL-1β optionally in combination with one or both of sTNFR1 and sTREM1 in EVLP perfusate are described. The methods involve for example, i. obtaining one or more test EVLP perfusate samples of a donor lung; ii. determining in one or more test EVLP perfusate sample of a donor lung, a polypeptide level of the at least three biomarkers selected from IL-8, IL-6, IL-10 and IL-1β and optionally one or both of sTNFR1 and sTREM1 i; and iii. a) comparing the one or more parameter values related to a level of the at least three biomarkers in the perfusate sample with control EVLP data or a cut-off level, wherein the differential level is indicative of outcome/risk of after transplant or of an EVLP outcome; or b) using the one or more parameter values related to a level of the at least three biomarkers in combination, as part of an algebraic calculation or model of outcome/risk.

This is a Patent Cooperation Treaty application which claims the benefit of 35 U.S.C. 119 based on the priority of U.S. Provisional Patent Application 62/779,220, filed on Dec. 13, 2018, and U.S. Provisional Patent Application 62/796,942, filed on Jan. 25, 2019, each of which is incorporated herein by reference in its entirety.

### FIELD

The disclosure pertains to methods of assessing perfusate samples of donor lung grafts that have been submitted to Ex Vivo Lung Perfusion, optionally in combination with other parameter values, for predicting patient outcome and for determining suitability for transplant.

### BACKGROUND

Ex vivo lung perfusion (EVLP) is a novel technique that was developed to prolong the normothermic assessment period of donor organs during lung transplantation (1-5). EVLP has been clinically validated and the technique is gaining widespread adoption worldwide. Currently, EVLP is hampered by a lack of predictive biomarkers that serve as reliable markers as to the process of EVLP, or the outcome of the organs that have been subject to EVLP during organ transplantation. Specifically, it is difficult to predict “patient outcome(s)” (PO) after transplant with lung having been subject to EVLP. Furthermore, many potential donor organs are placed on EVLP with the hope that they will improve and become suitable for transplant; however, in some cases the status of these lungs may not change and will ultimately be discarded following EVLP. The current selection of the healthiest donor lungs is based on a number of clinical findings including: donor type (brain and cardiac death donors), age, smoking history, arterial blood gasses (ABGs), chest radiograph and bronchoscopic findings, as well as physical examination by the physician (6-8). Clinicians are very conservative during this examination and selection process, which results in a poor organ utilization rates. Methods that identify which organs are suitable for transplant could aide clinical teams in the decision making processes surrounding for example the use and process of EVLP.

The addition of a proteomic-based approach to assess donor organs during and after the EVLP procedure could allow for one or both of (i) prediction/determination of PO post-transplant and (ii) stratification of lung response to EVLP (for example to determine the likelihood of lung not improving with EVLP).

During the EVLP procedure, an acellular perfusate solution is circulated throughout the circuit. Upon the initiation of EVLP, there are no detectable levels of polypeptides specific to the lung in the acellular solution. As the procedure progresses, polypeptides are flushed and/or secreted from the donor organ and begin to accumulate in the perfusate. Identification and quantification of the various polypeptides and associated levels might provide a measure of health for the donor organ, which could be used to stratify potential donor lungs based on predicted response to EVLP and/or predicted PO.

Previous studies have shown that perfusate-derived polypeptide levels during EVLP could differentiate lungs that are transplanted with PGD Grade 0/1 compared to those that develop PGD Grade 3 after transplantation (9).

At present, there are no gold-standards for EVLP assessments. For example, only PO₂ may be used for decision-making.

There is a need for the development of suitable biomarkers, kits, compositions, and scoring metrics for screening for, diagnosing, detecting or predicting (i) EVLP outcomes and/or (ii) screening for, diagnosing, detecting or predicting PO after transplantation of EVLP treated lungs.

### SUMMARY

The inventors have identified several biomarker parameters that are differentially detected in EVLP treated donor lungs that are associated with one or more patient outcomes (PO) in transplanted patients and/or donor lung suitability for transplant after EVLP. Specifically, the inventors have identified key polypeptides that are present in EVLP perfusate that can for example in combination, be used to determine suitable organs for transplantation and/or to predict various aspects of PO including intensive care unit (ICU)-related length of stay, which can be used to select organs for transplant and/or EVLP. In addition, the inventors have found that various biochemical and physiological parameters can improve the predictive power of the biomarkers described herein.

A proteomic-based or proteomic and biochemical/physiological scoring approach to assess donor organs during the EVLP procedure could allow for the determination of a score and/or cutoff levels that would indicate the overall quality of the organ. This could help guide physicians in determining for example (i) the suitability for transplant and/or (ii) the predicted response of the recipient post-transplantation.

The disclosure provides in an aspect, methods for predicting patient outcome (PO) risk.

One aspect of the present disclosure is a method for the screening, diagnosing, or detecting of an outcome/risk comprising:
i. determining, in one or more test EVLP perfusate sample(s) of a donor lung, one or more parameter values relating to a level of polypeptide of at least three biomarkers selected from IL-6, IL-8, IL-10 and IL-1β, optionally in combination with one or both of sTNFR1 and sTREM1; and
ii. a) comparing the one or more parameter values of each of the at least three biomarkers in the one or more perfusate sample with control EVLP data or a cut-off level, wherein a differential biomarker level is indicative of outcome/risk optionally after transplant; and/or
   b) using the one or more parameter values of the at least three biomarkers in combination, as part of an algebraic calculation or model of outcome/risk optionally after transplant.

The outcome/risk can for example be the risk of a negative post-lung transplant patient outcome such as extended ICU length of stay (e.g. number of days in ICU greater than or equal to 3), extended time on ventilator, extended post-transplant hospital stay etc.

The outcome/risk can for example be suitability for transplant, likelihood to be declined after EVLP or other parameter associated with suitability of the donor lung.

In an embodiment, the method further comprises determining one or more biochemical parameter values in the one or more test EVLP perfusate samples and/or determining one or more physiological parameter values of the donor lung, and comparing the determined parameter value(s) with control EVLP data or a cut-off level and/or optionally using the determined biochemical and/or physiologic parameter value(s) and the determined parameter values of the at least three biomarkers in combination, as part of an algebraic calculation or model of outcome/risk after transplant

In an embodiment, the method further comprises identifying a donor lung that has a decreased risk of having a negative post-lung transplant PO and optionally transplanting such donor lung into a suitable recipient.

In another embodiment, the method further comprises identifying a donor lung that has an increased risk of having a negative transplant outcome and optionally discarding the donor lung or using the donor lung for research or other purposes.

Another aspect of the present disclosure is a method for the early detection of a donor lung that will be declined at the end of the EVLP process comprising:
determining in one or more test EVLP perfusate sample of a donor lung one or more parameter values relating to a level of a polypeptide of at least three biomarkers selected from IL-6, IL-8, IL-10 and IL-1β, optionally in combination with one or both of sTNFR1 and sTREM1; and
   comparing the one or more parameter values of each of the at least three biomarkers in the perfusate sample and optionally the biochemical and/or physiologic parameter values, with control EVLP data or a cut-off level, wherein the differential level is indicative of a lung that will be accepted or declined for transplantation; and/or
   using the one or more parameter values of the at least three biomarkers in combination and optionally the biochemical and/or physiologic parameter values, as part of an algebraic calculation or model of suitability for transplantation.

In an embodiment, the method further comprises discarding the donor lung, and/or using the donor lung for research or other purposes if the at least three biomarker levels indicate that the lung will be declined for transplantation.

In an embodiment, the method further comprises determining one or more biochemical parameter values in the one or more test EVLP perfusate samples and/or determining one or more physiological parameter values of the donor lung, comparing the determined parameter(s) with control EVLP data or a cut-off level, and/or optionally using the determined parameter value(s) and the one or more parameter values related to a level of the at least three biomarkers in combination, as part of an algebraic calculation of suitability for transplantation or that a lung will be accepted or declined for transplantation.

In an embodiment, a computer-implemented method for the detecting of outcome/risk as it relates to donor includes
obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
querying, via one or more computers, a database of control EVLP data (e.g. known concentration data) or a cut-off level associated with patient outcome or transplant suitability to compare the EVLP data to the control EVLP data or cut-off level; and
determining a ranking score based on the comparison between the EVLP data and the control EVLP data or cut-off level.

In an embodiment, a system for the detecting of outcome/risk as it relates to donor lungs includes:
a first component that is configured to obtain EVLP data relating to one or more test EVLP perfusate samples of a donor lung, the EVLP data comprising one or more parameter values related to a level of polypeptide of at least three biomarkers selected from IL-6, IL-8, IL-10, and IL-1β, optionally in combination with one or both of sTNFR1 and sTREM1;
a second component that is configured to query a database of control EVLP data (e.g. known concentration data) or a cut-off level associated with patient outcome or transplant suitability to compare the EVLP data to the control EVLP data or cut-off level; and
a third component that is configured to determine a ranking score based on the comparison between the EVLP data and the control EVLP data or cut-off level.

In an embodiment, a non-transitory computer-readable storage medium storing computer-readable instructions includes:
instructions for obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
instructions for querying a database of control EVLP data (e.g. known concentration data) or a cut-off level associated with patient outcome or transplant suitability to compare the EVLP data to the control EVLP data or cut-off level; and
instructions for determining a ranking score based on the comparison between the EVLP data and the control EVLP data or cut-off level.

In an embodiment, a non-transitory computer-readable storage medium storing computer-readable instructions, includes:
instructions for obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
instructions for querying a database of control EVLP data (e.g. known concentration data) or a cut-off level associated with patient outcome or transplant suitability to compare the EVLP data to the control EVLP data or cut-off level; and
instructions for determining a ranking score based on the comparison between the EVLP data and the control EVLP data or cut-off level.

In an embodiment, a computer-program product for use in conjunction with an electronic device can include a non-transitory computer-readable storage medium and a computer-program mechanism embedded therein, the computer-program mechanism comprising:
instructions for obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
instructions for querying a database of control EVLP data (e.g. known concentration data) or a cut-off level associated with patient outcome or transplant suitability to compare the EVLP data to the control EVLP data or cut-off level; and
instructions for determining a ranking score based on the comparison between the EVLP data and the control EVLP data or cut-off level.

The disclosure also includes kits containing antibodies for the detection of the biomarkers of the invention that are used to measure the biomarker polypeptide levels.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIG. 1 shows a series of graphs depicting EVLP outcome predictive performance of a scoring metric (Version 1 (v1)). FIG. 1A) shows the comparison of transplanted and declined lungs when scored using v1 biomarker scoring metric (n=55). FIG. 1B) shows the corresponding area under the ROC curve for EVLP outcome prediction.

FIG. 2 shows a series of graphs depicting EVLP outcome predictive performance of the scoring metric of the present disclosure (Version 2 (v2)). FIG. 2A) shows the comparison of transplanted and declined lungs when scored using biomarker scoring metric v2 for the same clinical cases shown in FIG. 1 (n=55). FIG. 2B) shows the corresponding area under the ROC curve for EVLP outcome prediction.

FIG. 3. shows a series of graphs depicting recipient outcome predictive performance of the v1 scoring metric. FIG. 3A) shows the comparison of good (ICU <3 days) transplanted lungs and poor (ICU >3 days) lungs when scored using v1 of the biomarker scoring metric (n=21). FIG. 3B) shows the corresponding area under the ROC curve for recipient outcome prediction.

FIG. 4 shows a series of graphs depicting recipient outcome predictive performance of the present scoring metric v2. FIG. 4A) shows the comparison of good (ICU <3 days) transplanted lungs and poor (ICU >3 days) lungs when scored using v2 of the biomarker scoring metric for the same clinical cases shown in FIG. 3 (n=21). FIG. 4B) shows the corresponding area under the ROC curve for recipient outcome prediction.

FIG. 5 shows a series of graphs depicting EVLP Outcome Predictive Performance for a set of 6 markers. FIG. 5A) shows the comparison of transplanted and declined lungs when scored using IL-6, CXCL8, IL-10, IL-1β, sTNFR1, sTREM1 as biomarkers during clinical EVLP cases (n=32). FIG. 5B shows the corresponding area under the ROC curve for EVLP outcome prediction.

FIG. 6 is a series of graphs showing that combined models of biological, biochemical, and physiological parameters predict transplant outcome. 6A shows regression model results of a combined biological & biochemical model for the determination of those lungs suitable for transplantation. 6B shows the receiver operating characteristic for the combined biochemical and biological model. 6C shows regression model results of a combined biological & physiological model for the determination of those lungs likely to result in excellent (ICU ≤3 days) recipient outcomes. 6D shows the receiver operating characteristic for the combined model. p-values are indicated on each graph.

FIG. 7 illustrates a computer system for the screening, diagnosing and detecting of outcome/risk as it relates to donor lungs in accordance with one embodiment.

### DETAILED DESCRIPTION OF THE DISCLOSURE

### I. Definitions

The term “patient outcome” also referred to as “outcome” as used herein means one or more of primary graft dysfunction (PGD) grade, graft-related patient death, total hospital length of stay, transplant-related hospital length of stay, total intensive care unit (ICU) length of stay, transplant-related ICU length of stay, post-transplant ICU length of stay, APACHE score, days on mechanical ventilation, patient-related use of extracorporeal membrane oxygenation (ECMO).

The term “biomarkers of the invention” as used herein means three or more of IL-6 (also referred to as IL6), IL-8 (CXCL8), IL-10 (also referred to as IL10) and IL-1β (also referred to as IL1β or IL1beta), optionally wherein at least one is IL-1β, optionally in combination with one or both of sTNFR1 (soluble (TNFRSF1A)) and sTREM1 (soluble (TREM1)).

The term “biochemical parameter value” as used herein refers to a level or differential of a biochemical parameter measured in the EVLP, optionally the same perfusate sample, where said biochemical parameters can include, but are not limited to: base excess, bicarbonate, potassium, sodium, calcium, chloride, glucose, lactate, pH, etc. Base excess for example, is a number derived from the acid-base chemistry of the EVLP perfusate.

The term “physiological parameter value” as used herein refers to a measurement or differential of a physiological parameter of the donor lung, where said physiological parameters can include, but is not limited to: driving pressure, PCO₂ (measured and differential), PO₂ (also referred to as gas exchange and including measured and differential), airway pressure, static and dynamic compliance, PA and/or LA pressure, and/or pulmonary vascular resistance, etc. of the donor lung. These parameters can be measured using a ventilator, patient monitor (e.g. GE Dash 3000s connected to the lung/EVLP system and used to monitor pressures during EVLP), or calculated from the outputs of these machines (i.e., subtract two values).

The term “polypeptide” as used herein refers to a polymer consisting a number of amino acid residues bonded together in a chain. The polypeptide can form a part or the whole of a protein. The polypeptide may be arranged in a long, continuous and unbranched peptide chain. The polypeptide may also be arranged in a biologically functional way. The polypeptide may be folded into a specific three dimensional structure that confers it a defined activity. The term “polypeptide” as used herein is used interchangeably with the term “protein”.

The term “IL-6” or “IL6” as used herein means interleukin-6 which is a secreted cytokine, and includes all naturally occurring forms, for example from all species and particularly human including for example human IL-6 which has amino acid sequence accession P05231, herein incorporated by reference.

The term “IL-8” also referred to as CXCL8, as used herein means interleukin-8 which is a secreted cytokine, and includes all naturally occurring forms, for example from all species and particularly human including for example human IL-8 which has amino acid sequence accession P10145, herein incorporated by reference.

The term “IL-10” or “IL10” as used herein means interleukin-10, which is a secreted cytokine, and includes all naturally occurring forms, for example from all species and particularly human including for example human IL-10 which has amino acid sequence accession P22301, herein incorporated by reference.

The term “IL1β”, “IL-1β” or “IL1beta as used herein means interleukin-1β, which is a secreted cytokine, and includes all naturally occurring forms, for example from all species and particularly human including for example human IL-10 which has amino acid sequence accession P01584, herein incorporated by reference.

The term “sTNFR1” or “soluble (TNFRSF1A)” used herein means non-cell bound forms of tumor necrosis factor (TNF) receptor superfamily member 1A, and includes all naturally occurring cleaved or released forms, for example from all species and particularly human including for example human sTNFR1 which has at least the extracellular portion of TNFR1, for example amino acid 22 to 211 of accession number P19438, herein incorporated by reference.

The term “soluble TREM1” or sTREM-1 as used herein means non-cell bound forms of Triggering receptor expressed on myeloid cells and includes all naturally occurring cleaved or released forms, for example from all species and particularly human including for example human sTREM-1 which has at least the extracellular portion of sTREM-1, for example amino acid 21 to 205 of accession number Q9NP99, herein incorporated by reference.

The term “donation after cardiac death” or “DCD” as used herein means the withdrawal of life support of a patient after it has been determined that there is no long-term prognosis for recovery, and subjects who experience cardiocirculatory arrest and a qualified decision is made to terminate or not initiate resuscitation. A DCD lung graft is accordingly a lung graft obtained from such a patient. DCD is also meant to include NPOD (non-perfused organ donor) and uDCD (uncontrolled (Maastricht) DCD) donors.

The term “donation after brain death” or “DBD” as used herein means donors who experience the irreversible end of all brain activity but whose body, including transplantable organs, are maintained through external mechanical means.

The terms “control EVLP data” and “cut-off level” as used herein refer to a comparator value or set of values of EVLP data for a graft or grafts with known outcome (to which test donor organ EVLP data can be compared) and a predetermined or selected threshold value based on a plurality of known outcome grafts and a desired specificity and/or selectivity. For example, for biomarkers associated with increased polypeptide level in poor grafts e.g. those with poor PO or which are likely to be declined following EVLP, above a selected threshold, a graft is identified as having an increased risk of developing poor PO post-transplant and/or being declined after EVLP and below which (and/or comparable to) a candidate donor lung is identified as having a decreased risk of developing poor PO post-transplant or being declined post EVLP. The threshold value can for example for each of the one or more polypeptide biomarkers of the invention, be determined from the parameter values related thereto of the biomarkers in a plurality of known outcome lungs, as well as include biochemical and/or physiologic parameter values. For example, an optimal or an acceptable threshold can be selected based on the desired tolerable level of risk. The control and/or the cut-off level may for example depend on the PO being assessed. For example, for the patient outcome “total ICU stay”, patients that have pre-transplant ICU stay are typically sicker and would also have longer post-transplant ICU stays. The cut-off level may also for example include donor characteristics, for example gender, type (DBD or DCD), age, body mass index (BMI), and/or smoking history as well as biochemical and/or physiologic parameter values. Accordingly, the biomarker ‘cut-off’ can in some embodiments be adjusted for patients who have different duration of ICU stays, and donor characteristics.

The term “reference score” means a calculated score based on control lungs with known outcome, the score considering for example levels of the at least three biomarkers and optionally biochemical and/or physiological parameter values as well as donor data such as donor characteristics. The reference score can be a reference PO score or a reference transplant suitability, to which an injury score is compared.

The term “good outcome lung grafts” as used herein means lung grafts that are predicted to be and/or which are characterized as being suitable for clinical transplantation after EVLP and/or which result in a good PO in the recipient after transplantation. For example, good PO could include being free from: graft-related death causes within 30 days, PGD3, extracorporeal life support/ECMO, prolonged hospital/ICU stays (for example, prolonged ICU stay can be greater than at least 3 days, for example greater than two weeks (14 days)) or prolonged time spent on a mechanical ventilator. An ICU stay of 3 days or less can be considered and excellent outcome lung graft.

The term “poor outcome lung grafts” or “poor PO” as used herein means lung grafts that are predicted to be and/or which are characterized as being less or unsuitable for clinical transplantation after EVLP or, in the recipient after transplantation, inducing poor PO such as death from graft-related causes within 30 days, PGD3, requiring extracorporeal life support/ECMO, prolonged hospital/ICU stays, or time on mechanical ventilation. Examples of a poor-PO graft include a graft that after transplanting would result in a patient requiring an extended ICU stay (for example greater than 3 days or greater than two-weeks (14 days)), as well as a graft that has an increased risk of having a PGD3 lung transplant outcome. A lung graft can be characterized as being unsuitable for clinical transplant after EVLP for example after visual and physiological examination such as when gas exchange function is not acceptable represented by a partial pressure of oxygen less than 350 mmHg with a fraction of inspired oxygen of 100%; or 15% worsening of lung compliance compared to 1 h EVLP; or 15% worsening of pulmonary vascular resistance compared to 1 h EVLP; or worsening of ex vivo x-ray. The assessment of suitability for transplant requires significant skill and experience. Biomarkers that are able to predict suitability can provide a more accessible quantitative benchmark for use in assessing transplant suitability.

The term “Acute Physiology And Chronic Health Evaluation Score” or “APACHE score” as used herein refers to an initial risk classification system for severely ill hospitalized patients. For example, it is applied within 24 hours of admission of a patient to an ICU. An integer score is computed based on several measurements, and higher scores correspond to more severe disease and a higher risk of death. For example, the point score is calculated from a patient's age and 12 routine physiological measurements: AaDO₂ or PaO₂ (depending on FiO2); temperature (rectal); mean arterial pressure; pH arterial; heart rate; respiratory rate; sodium (serum); potassium (serum); creatinine hematocrit; white blood cell count; and Glasgow Coma Scale. The score can also take into account of whether the patient has acute renal failure, and whether prior to hospital admission the patient has severe organ system insufficiency or is immunocompromised.

The term “perfusate sample” as used herein means an aliquot of a perfusion solution such as STEEN Solution™ that is used for EVLP and which is taken subsequent to starting EVLP, for example after at least or at about 15, 30, 45, 60, 75, 90 and/or 105 min, and/or after at least or at about 2, 2.5, 3, 3.5, 4, 4.5, 5, 5.5 and/or 6 hours subsequent to starting EVLP, or optionally at time of fluid replenishment or any time between 15 min and 6 hours, optionally between 1 hour and 4 hours, or any increment of 1 minute, 5 minutes or 15 minutes between 0 and 6 hours or any time therebetween. The term “perfusate sample” and “EVLP perfusate sample” are used interchangeably in the present disclosure. Perfusate samples can be used directly or snap frozen for later testing. The perfusate sample can, for example, be purified and/or treated prior to assessment.

The term “perfusion solution” as used herein means a buffered nutrient solution that can be used for EVLP, including for example STEEN Solution™. STEEN Solution™ is a buffered extracellular solution developed specially for EVLP that contains Dextran 40, human serum albumin and extracellular electrolyte composition (low K+) that provides cellular/organ protection and optimized colloid osmotic pressure. The skilled person can readily recognize that the perfusion solution can be any buffered nutrient solution that is suitable for and/or supports ex vivo lung perfusion for lungs that may be used for transplantation.

The term “declined lungs” as used herein means lungs that after EVLP are declined for transplant. Such lungs can be discarded and/or used for research or other purposes. Lungs are presently typically declined for example if gas exchange function is not acceptable, represented by a partial pressure of oxygen less than 350 mmHg with a fraction of inspired oxygen of 100%; or 15% worsening of lung compliance compared to 1 h EVLP; or 15% worsening of pulmonary vascular resistance compared to 1 h EVLP; or development of significant edema; or worsening of ex vivo x-ray. As described herein, lungs are declined during or at the end of the EVLP process if comparison between parameter values related to polypeptide levels of biomarkers from EVLP perfusate samples of a donor lung with a control or cut-off level determines that the donor lung is to be declined for transplantation. The biomarker combination can be at least three biomarkers described herein, including the combination of polypeptides IL-8, IL-6, IL-10 and IL-1β, preferably wherein the combination includes at least IL-1β, and optionally wherein the combination of polypeptides includes one or both of sTNFR1 and sTREM1 in the test EVLP perfusate samples. The assessment can involve the generation of transplant suitability score for the donor lung based on the parameter values derived from the polypeptide levels of the three or more biomarkers.

The term “suitability for transplant” as used herein means an organ that is predicted to be a good PO lung graft, for example to have a decreased risk of a prolonged ICU (e.g. greater than 3 days, greater than 14 days) stay post-transplant. For example, a lung that would be predicted to involve 3 days or less of ICU stay for the recipient, would be considered a particularly suitable lung for transplant. A lung that would be predicted to involve 14 days or less of ICU stay for the recipient, may be considered a suitable lung for transplant.

The term “PGD3” as used herein means Primary Graft Dysfunction Grade 3 as defined by the standardized consensus criteria of International Society for Heart and Lung Transplantation (ISHLT) or similar.

The term “antibody” as used herein is intended to include monoclonal antibodies including chimeric and humanized monoclonal antibodies, polyclonal antibodies, humanized antibodies, human antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques. The skilled person can readily recognize that a suitable antibody for the invention is any antibody useful for detecting biomarkers described herein in any detection method described herein. For example, useful antibodies include antibodies that specifically bind to IL-6, IL-8, IL-10 or IL-1β polypeptide.

The term “detection agent” refers to an agent (optionally a detection antibody) or method (e.g., mass spectrometry) that selectively binds and is capable of binding its cognate biomarker compared to another molecule and which can be used to detect a level and/or the presence of the biomarker. A biomarker specific detection agent can include probes and the like as well as binding polypeptides such as antibodies which can for example be used with immunohistochemistry (IHC), Luminex® based assays, ELISA, immunofluorescence, radioimmunoassay, dot blotting, FACS, protein microarray, Western blots, immunoprecipitation followed by SDS-PAGE immunocytochemistry, other immunoassays and platforms (e.g., Simple Plex assay (Protein Simple)) as well as others. Mass spectrometry methods can also be used to detect one or more parameter values related to a level of a biomarker described herein. Similarly, “an antibody or fragment thereof” (e.g. binding fragment), that specifically binds a biomarker refers to an antibody or fragment that selectively binds its cognate biomarker compared to another molecule. “Selective” is used contextually, to characterize the binding properties of an antibody. An antibody that binds specifically or selectively to a given biomarker or epitope thereof will bind to that biomarker and/or epitope either with greater avidity or with more specificity, relative to other, different molecules. For example, the antibody can bind 3-5, 5-7, 7-10, 10-15, 5-15, or 5-30 fold more efficiently to its cognate biomarker compared to another molecule. The “detection agent” can for example be coupled to or labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “determining” as used herein includes for example measuring a level such as a concentration, and/or obtaining measured data.

The term “EVLP data” as used herein may refer to data related to an EVLP procedure, including, for example: data related to patient and patient outcome; biomarkers including IL-8, IL-6, IL-10 and IL-1β, sTNFR1, sTREM1 (e.g. levels, rates etc); biochemical parameter values including base excess, bicarbonate, potassium, sodium, calcium, chloride, glucose, lactate and pH and; physiological parameter values including driving pressure, PCO₂ (measured and differential), PO₂ (measured and differential), airway pressure, static and dynamic compliance, PA and/or LA pressure, and pulmonary vascular resistance; data related to features and timing of the EVLP procedure, including time after start of EVLP perfusate sample obtained e.g Early EVLP (such as EVLP for 1 hour) or Late EVLP (such as EVLP for 4 hours); and donor data.

The term “donor data” can refer to for example but not limited to donor characteristics, for example gender, type (DBD or DCD), age, body mass index (BMI), and/or smoking history.

The term “parameter value related to a level” or “level” as used herein interchangeably in relation to a biomarker refer to an amount (e.g. relative amount or concentration as well as parameter values calculable based thereon such as a rate or ratio) of biomarker (i.e. polypeptide related level) that is detectable, measurable or quantifiable in a test biological sample and/or a reference biological sample, for example, a test perfusate sample and/or a reference perfusate sample. For example, the parameter value related to a level can be a rate such as pg/mL/hour, a concentration such as μg/L, ng/mL or pg/mL, a relative amount or ratio such as 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10, 15, 20, 25, and/or 30 times more or less than a control biomarker or reference profile level. The control biomarker polypeptide level can, for example, be the average or median level in a plurality of known outcome lungs. Parameter values related to a level include concentration, rate of production and a ratio or fold increase (e.g. concentration at a later time point (such as 4 hours) divided by a concentration at an earlier time point for the same biomarker). The parameter values are EVLP data, for example from a test perfusate, and can also include the time after starting EVLP the perfusate sample was taken.

The term “subject” as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure, are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods

Disclosed herein are polypeptide biomarkers that can be used to assess whether a lung graft such as a marginal lung graft that is subjected to normothermic ex vivo lung perfusion (EVLP), is suitable for transplant.

The inventors have identified several biomarkers that are differentially expressed in donor lungs that are associated with, and can be used to predict patient outcomes (PO) post-transplant. Specifically, the inventors have identified key polypeptides that are present in EVLP perfusate that predict intensive care unit (ICU) length of stay and/or a declined donor organ. The disclosure provides in an aspect, methods for diagnosing patient risk and identifying declined lungs early in the EVLP process.

It is also demonstrated herein that the predictiveness of the biomarker levels can be enhanced by including one or more biochemical or physiological parameters.

One aspect of the present disclosure is a method for the screening, diagnosing, or detecting the outcome/risk as it relates to donor lungs such as a risk of a prolonged ICU stay comprising:
i. determining in one or more test EVLP perfusate samples of a donor lung one or more parameter values related to a level of three or more biomarkers selected from IL-6, IL-8, IL-10 and IL-1β, optionally in combination with one or both of sTNFR1 and sTREM1;
ii. optionally determining in one or more test EVLP perfusate samples of the donor lung, one or more biochemical parameter values and/or determining one or more physiological parameter values of the donor lung,
iii. comparing the one or more parameter values related to a level of each the at least three biomarkers in the perfusate sample with control EVLP data or a cut-off level, wherein the differential biomarker level is indicative of the outcome/risk optionally after transplant; and/or optionally
iv. using the one or more parameter values related to a level of the at least three biomarkers in combination, and optionally the more or more biochemical and/or physiological parameter value(s) as part of an algebraic calculation or model of outcome/risk optionally after transplant.

The outcome/risk can for example be suitability for transplant or an after transplant outcome risk such as PGD, long ICU stay or length of stay (e.g. hospital stay).

Where an algebraic calculation is performed, the output of the calculation, e.g. an injury score, e.g. a PO-score or transplant suitability score, can be compared to a reference score.

In some embodiments, the method comprises comparing the measured levels of the at least three biomarkers to known levels, optionally by querying a database of known levels, that are associated with transplant outcome (ex. PGD status, ICU LOS) and using the one or more parameter values related to a level of the at least three biomarkers in combination, as part of an algebraic calculation or model to arrive at an injury score indicative of for example outcome/risk after transplant. The injury score can be compared to a reference score to determine if the lung is at risk of post graft dysfunction, long ICU stay or long hospital stay (LOS=length of stay).

Also provided in another aspect a method for predicting a patient outcome (PO) variable for a lung transplant recipient after transplant, the method comprising:
i. obtaining one or more test EVLP perfusate samples of a perfusion solution collected during perfusion of a donor lung;
ii. measuring in the one or more test EVLP perfusate samples one or more parameter values related to a level of at least three biomarkers selected from IL-8, IL-6, IL-10 and IL-1β, optionally in combination with one or both of sTNFR1 and sTREM1;
iii. optionally determining in one or more test EVLP perfusate samples of the donor lung, one or more biochemical parameter values and/or determining one or more physiological parameter values of the donor lung;
iv. optionally generating a PO variable score for the donor lung based on the one or more parameter values related to a level of at least three biomarkers and optionally the one or more biochemical parameter values and/or physiological parameter values; and
v. comparing the one or more parameter values or optionally the PO variable score for the donor lung with control EVLP data or a cut-off level or reference score, wherein the PO variable score is indicative of a PO variable after transplant.

In some embodiments, the method further comprises transplanting the donor lung if the donor lung has a desirable outcome/risk, subjecting the donor lung to further EVLP or other treatment and subsequently reassessing the donor lung outcome/risk if the donor lung has an outcome/risk below a selected threshold. In other embodiments, the method further comprises selecting a recipient considering the outcome/risk.

In an embodiment, the outcome/risk after transplant (also referred to as patient outcome related risk) or PO variable is selected from ICU length of stay, post-transplant hospital length of stay, number of days on a ventilator, APACHE score and post graft dysfunction (PGD) grade, optionally PGD0/1 or PGD3.

In an embodiment, the outcome/risk is determined to be acceptable or sufficient to discontinue EVLP.

In an embodiment, the outcome/risk or PO variable is total ICU stay.

In an embodiment, the outcome/risk or PO variable is ICU post-transplant length of stay. For example, this can be used to predict likelihood of short ICU post-transplant stay, e.g. 3 days or less, or long ICU post-transplant stay, e.g. 6 days or longer.

In another embodiment, the outcome/risk or PO variable is post-transplant hospital length of stay.

In yet another embodiment, the outcome/risk or PO variable is number of days the recipient post-transplant on a ventilator.

In another embodiment, the outcome/risk or PO variable is APACHE score.

In another embodiment, the outcome/risk or PO variable is post graft dysfunction (PGD), optionally PGD0/1 or PGD3.

In an embodiment, the method further comprises identifying or selecting donor lungs that have a decreased risk of having poor PO post lung transplant. In an embodiment, the donor lung is selected, prepared for and optionally transplanted into a suitable recipient if the outcome/risk or PO variable or transplant suitability is acceptable, for example when compared to a selected level or score. For example, the donor lung is suitable, if the one or more parameter values related to a level, combination of parameter values, injury score e.g. PO-score or transplant suitability score, are indicative of positive outcome, e.g. below the selected cut-off value or reference score.

The algebraic calculation, injury score e.g. PO-score or transplant suitability score, can be calculated using a computer. In one embodiment, the algebraic calculation comprises the product of the logarithm transformed concentration of each the at least three biomarkers.

The algebraic calculation, PO-score or transplant suitability score can then be compared to a control, cut-off value, or reference score, to predict patient outcome and or transplant suitability. The comparison can for example be to control EVLP data or a cut-off value in a database optionally as further described herein.

The database comprises for example the various parameters and levels described herein as well as outcome risk data. The database may continually be updated to include additional cases and outcome data.

The database can comprise a plurality of EVLP control data, e.g. parameter values, donor data and transplant patient outcome data, as well as scores, optionally ranges and the like from for example for at least or about 100, 200, 300 or more donor/patients.

The reference score can also for example be a range, wherein a range of scores indicate relative suitability. Depending on the recipient status, different ranges can identify when an organ is suitable for transplant into a particular recipient.

In another embodiment, the method further comprises identifying a donor lung that has an increased risk of having a negative transplant outcomes and optionally discarding the donor lung or using the donor lungs for research or other purposes if the outcome/risk or PO variable is unacceptable or above the cut-off value. For example, the donor lung is declined, if the one or more parameter values related to a level, combination of parameter values or injury score is/are indicative of poor outcome, e.g. is/are above the cut-off value.

The combination of the parameter values is optionally a linear combination. The combination of parameter values is optionally a non-linear combination.

A further aspect relates to early detection of donor lungs that will be ultimately declined after EVLP. Clinical EVLP is typically approximately 4 to 6 hours. As demonstrated herein, the inventors have identified biomarker parameter values that can identify donor lungs that would likely be declined after EVLP earlier in the EVLP process, for example after at least or at about the 1 hour mark. The earlier identification is a benefit for example in time and resources.

Accordingly another aspect of the present disclosure is a method for the early detection of donor lungs that will be declined at the end of the EVLP process comprising:
obtaining one or more test EVLP perfusate samples of a perfusion EVLP solution collected during perfusion of the donor lung;
determining in the one or more test EVLP perfusate samples the one or more parameter values related to a level of at least three biomarkers selected from IL-6, IL-8, IL-10 and IL-1β, optionally in combination with one or both of sTNFR1 and sTREM1;
optionally determining in one or more test EVLP perfusate samples of the donor lung, one or more biochemical parameter values and/or determining one or more physiological parameter values of the donor lung; and
   comparing the one or more parameter values related to a level of each of the at least three biomarkers in the perfusate sample and optionally the biochemical and/or physiologic parameter values with control EVLP data or a cut-off, wherein the differential biomarker level is indicative of a lung that will be declined or accepted for transplantation; and/or
   using the one or more parameter values related to a level of the at least three biomarkers in combination and optionally the more or more biochemical and/or physiological parameter value(s), as part of an algebraic calculation or model to identify donor lungs that will be declined or accepted for transplantation.

In an embodiment, the method comprises:
obtaining one or more test EVLP perfusate samples of a perfusion EVLP solution collected during perfusion of the donor lung;
determining one or more parameter values related to a level of at least three biomarkers selected from IL-8, IL-6, IL-10 and IL-1β, and optionally one or both of sTNFR1 and sTREM1, in the one or more test EVLP perfusate samples and optionally one or more biochemical parameter values in said samples and/or physiological parameter values relating to said donor lung;
optionally generating a transplant suitability score for the donor lung based on the one or more parameter values related to a level of at least three biomarkers and optionally the one or more biochemical parameter values and/or physiological parameter values;
comparing the one or more parameter values or optionally the transplant suitability score for the donor lung with control EVLP data or a cut-off level or reference score, and
optionally continuing perfusion if the one or more parameter values or transplant suitability score indicates that the donor lung is suitable or may be suitable for transplantation and discontinuing perfusion if the one or more parameter values or the transplant suitability score indicates that the donor lung will be declined for transplantation.

For example, a lung may be suitable for transplant and perfusion may be continued to improve patient outcome. In some embodiments, instead of continuing perfusion, the donor lung may be selected for transplant and optionally transplanted into a recipient.

The one or more parameters compared or used in an algebraic formula or model, can include biomarker as well as biochemical and/or physiologic parameter values.

In another embodiment, the method further comprises discarding the donor lung and/or using the donor lung for research or other purposes if the at least three biomarker levels, or injury score, e.g. transplant suitability score, based thereon and optionally one or more parameter values, indicate that the lung will be declined for transplantation after EVLP.

A further aspect of the disclosure includes a method of selecting a donor lung for transplant, the method comprising:
determining in a test EVLP perfusate sample of a donor lung the one or more parameter values related to a level of a polypeptide of at least three biomarkers selected from IL-6, IL-8, IL-10 and IL-1β, optionally in combination with one or both of sTNFR1 and sTREM1;
optionally determining in one or more test EVLP perfusate samples of the donor lung, one or more biochemical parameter values and/or determining one or more physiological parameter values of the donor lung; and
comparing the one or more parameter values related to a level of each of the at least three biomarkers in the perfusate sample with control EVLP data or a cut-off level or as part of an algebraic formula, optionally where the algebraic formula also comprises the one or more parameters,
selecting the donor lung for transplant according to the one or more parameter values related to a level(s) of the at least three biomarkers in the test EVLP perfusate sample and optionally one or more parameters.

In an embodiment, the method comprises:
obtaining one or more test EVLP perfusate samples of a perfusion solution collected during perfusion of the donor lung;
determining one or more parameter values related to a level of at least three biomarkers selected from IL-8, IL-6, IL-10 and IL-1β and optionally one or both of sTNFR1 and sTREM1, in the one or more test EVLP perfusate samples;
optionally determining in one or more test EVLP perfusate samples of the donor lung, one or more biochemical parameter values and/or determining one or more physiological parameter values of the donor lung;
optionally generating a transplant suitability score for the donor lung based on the one or more parameter values of the at least 3 biomarkers and optionally the one or more biochemical and/or physiological parameter values;
comparing the one or more parameter values or optionally the transplant suitability score for the donor lung with control EVLP data or a cut-off level or reference score;
selecting the donor lung for transplant according to the one or more parameter values or the transplant suitability score.

In one embodiment, the one or more parameter values comprises a concentration of the at least three biomarkers of the invention.

The one or more parameter values for the at least three biomarkers, can be measured in samples taken at different time points. For example, 11-6 and IL-8 parameter(s) can be measured at 4 hours, and the TNFR1 parameter at 1 hour. They can also be subsequent samples. Further different parameter values can be combined e.g. concentration, rate or ratio of production during a selected interval of EVLP.

The biomarker parameter values and the biochemical and physiologic parameter values can be determined in any order and at different time points after EVLP.

In one embodiment, one or more parameters values comprises a rate of biomarker production of the at least three biomarkers.

In another embodiment, the method comprises determining in one or more test EVLP perfusate samples of the donor lung, one or more biochemical parameter values and/or determining one or more physiological parameter values of the donor lung.

In some embodiments, the perfusate sample or where multiple samples are assessed, at least one of the perfusate samples, used to determine the one or more parameter values related to a level of at least three biomarkers is also used to determine the one or more biochemical parameter values.

In other embodiments, different perfusate samples are used, for example a second perfusate sample, subsequently obtained can be assessed for the one or more biochemical parameter values

In some embodiments, the at least three biomarkers, preferably include IL-1β.

In one embodiment, the one or more parameters values comprises a ratio or fold polypeptide level of the concentration of the at least three biomarkers, wherein the ratio or fold polypeptide level is the concentration of a subsequent perfusate sample divided by the concentration of an earlier perfusate sample.

In one embodiment, the one or more parameter values comprises a concentration of the at least three biomarkers of the invention normalized to lung size (e.g. measured or predicted total lung capacity (TLC)). In one embodiment, the one or more parameter values comprise a concentration of the at least three biomarkers normalized to TLC. In one embodiment, the TLC is measured or determined based on gender and size of donor or optionally weight of the donor lung. In one embodiment, the TLC is measured or determined based on gender and height of donor.

As indicated herein an algebraic formula can be used.

In some embodiments, the algebraic formula/calculation is the product of the logarithm of each of the concentrations of the at least three biomarkers. In an embodiment, the logarithm is the natural logarithm.

Extrapolation based on perfusate levels measured during earlier time points can be useful for determining a parameter value of a concentration of a biomarker of the invention. Linear regression analysis can be used for example when assessing independent variables (e.g. polypeptide level of a biomarker and time) to predict an outcome (i.e. level of a biomarker in a future time). For example, values (e.g. 2 or more) collected from for example after 15 minutes, 30 minutes, 45 minutes, 1 hour or after 75 minutes after starting EVLP can be extrapolated to predict values expected at about 4 hours as determined for example by linear regression analysis. In one embodiment, the one or more parameter values comprises a concentration of each of the at least three biomarkers of the invention that has been extrapolated based on the perfusate levels measured during earlier time points. In some embodiments, the extrapolation is carried out with a linear regression model.

Logistic regression analysis is useful for univariate or multivariate analysis where the outcome has only a limited number of possible values. The skilled person in the art can readily recognize that logistic regression analysis is useful when the response variable is categorical in nature, such as to proceed with transplant or not. In an embodiment, ELVP outcome or patient outcome is predicted by logistic regression analysis.

A dataset can be provided including EVLP data, biochemical data, biological data or physiological data. Please note that the dataset can include any other parameters discussed in the present subject matter. The dataset can be applied to a regression model. The regression model can be trainable by a machine learning model or algorithm using the dataset or any one of: EVLP data, biochemical data, biological data or physiological data. For example, the regression model can be selected from a plurality of regression models including linear regression, polynomial regression, logistic regression, quantile regression, and ridge regression.

For example, the step of creating a regression model can include combining biological data, biochemical data and/or physiological data. It further includes processing the combined data to determine one or more feature points associated with a transplant outcome and to generate one or more feature vectors based on the one or more feature points. For example, this step can be repeated for a plurality of samples of the dataset or for a plurality of datasets. The step of creating the regression model further includes correlating the one or more features vectors obtained by performing the above steps and transplant/patient outcomes so as to create the regression model.

Furthermore, the regression model can be trained with one or more feature vectors using a machine learning based method.

In some embodiments, the method further comprises determining one or more biochemical parameters in the one or more test EVLP perfusate samples and/or one or more physiological parameters of the donor lung, comparing the measured parameter(s) values with control EVLP data or a cut-off level or reference score, wherein a differential biochemical and/or differential physiological level is indicative of outcome/risk after transplant and using the measured parameter(s) value(s) and the one or more parameter values related to a level of the at least three biomarkers in combination, as part of an algebraic calculation or model of outcome/risk after transplant.

Different biochemical parameters can be measured. For example, the one or more biochemical parameters can be selected from base excess, bicarbonate, potassium, sodium, calcium, chloride, glucose, lactate, pH, etc. Any one of the foregoing or a combination can be assessed.

Where one or more biochemical and physiological parameters are measured, these can be considered in the calculation of the injury score e.g. PO-score or transplant suitability score.

For example, logistic regression can be used to identify the coefficients assigned to the various input parameters (eg. biological, biochemical, physiological). During a given case, the values of the input parameters are measured and then entered into the defined equation that has the output of an injury score.

In addition to logistic regression additional methods (e.g. Al-based) can be used to mathematically model this situation (eg. neural networks, etc).

Different physiological parameters can be determined/obtained. For example, the one or more physiological parameters can be selected from driving pressure, PCO₂ (measured and differential), PO₂ (measured and differential), airway pressure, static and dynamic compliance, pulmonary artery (PA) and/or left atrium (LA) pressure, and/or pulmonary vascular resistance, etc

Any of the above parameters can be measured throughout the EVLP procedure (e.g. any time point after 0 and up to for example 6 hours) and can be measured directly. For example, the physiological parameters can be measured for example by pressure readings off of a ventilator/patient/pressure monitor, or an ABG machine, or calculated (for example, driving pressure). Biochemical and biomarker levels can be measured in the perfusate sample. For example, the biomarker protein levels can be measured using ELISA, and biochemical parameters such as sodium ion levels can be measured using ABG machine.

Some blood gas analyzers have the capability to measure electrolytes, glucose, lactate, CO-oximetry, etc. In some instances, an operator could measure these parameters (eg. electrolytes) separately.

It is known that the size of a subject right lung is generally larger than the size of left lung in an individual. The approximate ratio of size of right/left lung is 60:40.

In one embodiment, the one or more parameter value comprises a concentration of the at least three biomarkers of the invention that has been derived from a single right lung, single left lung, or combination thereof. In another embodiment, the one or more parameter value is adjusted based on if it is a right lung or a left lung.

The one or more parameter values can be corrected for various clinical (ex. donor lung size) and technical factors (ex. circuit dilution). Values can be normalized prior to determining a combinatorial score indicative of the health of the organ (e.g. suitability for transplant, increased likelihood of positive patient outcome).

The combination of markers is typically tested in the same sample although multiple samples taken at the same time or different times may be tested.

Normalization is used for example when inter-case comparison is to be made (i.e. between two different lungs); because lungs may be of different sizes and/or if dilution of the circuit is modified.

In one embodiment, the one or more parameter value is used to predict PO or transplant suitability, wherein the prediction can be substratified based on donor characteristics, for example, gender, type (DBD or DCD), age, body mass index (BMI), and/or smoking history. When donor characteristics are considered, the score determined may be penalized for certain characteristics such as smoking history.

In some embodiments, the at least three biomarkers comprise or are IL-6, IL-8, and IL-10.

In some embodiments, the at least three biomarkers comprise or are IL-6, IL-8, and IL-1β.

In some embodiments, the at least three biomarkers comprise or are IL-8, IL-10, and IL-1β.

In some embodiments, the at least three biomarkers comprise or are IL-6, IL-10, and IL-1β.

In some embodiments, the at least three biomarkers in the perfusate sample are IL-6, IL-8, IL-10, and IL-1β.

In some embodiments, the at least three biomarkers comprise or are IL-6, IL-8, sTNFR1 and IL-10.

In some embodiments, the at least three biomarkers comprise or are IL-6, IL-8, sTNFR1 and IL-1β.

In some embodiments, the at least three biomarkers comprise or are IL-8, IL-10, sTNFR1 and IL-1β.

In some embodiments, the at least three biomarkers comprise or are IL-6, IL-10, sTNFR1 and IL-1β.

In some embodiments, the at least three biomarkers in the perfusate sample are IL-6, IL-8, IL-10, sTNFR1 and IL-1β.

In some embodiments, the at least three biomarkers comprise or are IL-6, IL-8, sTREM1 and IL-10.

In some embodiments, the at least three biomarkers comprise or are IL-6, IL-8, sTREM1 and IL-1β.

In some embodiments, the at least three biomarkers comprise or are IL-8, IL-10, sTREM1 and IL-1β.

In some embodiments, the at least three biomarkers comprise or are IL-6, IL-10, sTREM1 and IL-1β.

In some embodiments, the at least three biomarkers in the perfusate sample are IL-6, IL-8, IL-10, sTREM1 and IL-1β.

In some embodiments, the at least three biomarkers comprise or are IL-6, IL-8, sTNFR1, sTREM1 and IL-10.

In some embodiments, the at least three biomarkers comprise or are IL-6, IL-8, sTNFR1, sTREM1 and IL-1β.

In some embodiments, the at least three biomarkers comprise or are IL-8, IL-10, sTNFR1, sTREM1 and IL-1β.

In some embodiments, the at least three biomarkers comprise or are IL-6, IL-10, sTNFR1, sTREM1 and IL-1β.

In some embodiments, the at least three biomarkers in the perfusate sample are IL-6, IL-8, IL-10, sTNFR1, sTREM1 and IL-1β.

Different combinations of markers can be used in any of the methods described herein.

In some embodiments, three biomarkers selected from IL-8, IL-6, IL-10 and IL-1β are assessed. In yet other embodiments, four biomarkers selected from IL-8, IL-6, IL-10 and IL-1β are assessed. In some embodiments, one or both of sTNFR1 and sTREM1 are assessed in addition to at least biomarkers selected from IL-8, IL-6, IL-10 and IL-1β.

In some embodiments, the biochemical parameters are or comprise pH, glucose, lactate, bicarbonate, sodium, potassium, calcium, chloride, and/or base excess levels.

In some embodiments, the physiological parameters are or comprise PO₂, PCO₂, static and/or dynamic compliance, airway pressure (eg. peak, plateau, mean), PA and/or LA pressure, driving pressure, and/or pulmonary vascular resistance measurements.

In some embodiments, the method includes first collecting a test EVLP perfusate sample.

EVLP involves mechanical ventilation and pumping a nutrient (i.e. perfusion) solution such as STEEN Solution™ (or equivalent) through the blood vessels of the lungs while at the same time supplying deoxygenated gas. An aliquot of the perfusion solution that has been pumped for a selected amount can be taken while the lung continues to be on the ventilator machine or an aliquot can be taken at the end of the EVLP process.

The biomarkers described herein can be measured in test EVLP perfusate samples.

In some embodiments, the perfusate samples are collected from the EVLP circuit. In some embodiment, the perfusate samples are collected directly from the left atrium or lung outflow, and/or pulmonary artery or lung inflow. In some embodiments, the perfusate samples are further tested or assayed without further processing. In other embodiments, the perfusate samples are diluted, separated, for example by centrifugation and/or concentrated prior to assaying polypeptide levels. In some embodiments, the perfusate samples are stored, for example frozen for example at −20° C.

Accordingly, in some embodiments, the method first comprises:
i. inserting the donor lung into a perfusion machine;
ii. using the perfusion machine to perfuse the donor lung with an EVLP solution; wherein the one or more test EVLP perfusate samples are obtained from the EVLP solution during perfusion of the donor lung.

A perfusion machine comprises various components including a sterile plastic dome for encasing the lung, which is attached to a ventilator, perfusion pump, gas exchange membrane, and comprises a sampling reservoir/apparatus for removing perfusate samples from the circuit. During the process, lung function can be evaluated continuously for various biochemical and physiological parameters. For example, the ventilator/patient monitor can monitor physiological parameters, providing if desired a continuous readout for or more of such parameters. Biomarker, biochemical and gasses can be measured in the perfusate.

Typically, a lung graft receives EVLP for about 4 to 6 hours. In an embodiment, the test EVLP perfusate sample is collected during EVLP, for example at desired time points where perfusate fluid is removed from the circuit, such as after at least or at about 15 min, at least or at about 30 min, at least or at about 45 min, after at least or at about 1 hour, after at least or at about 1.5 hours, after at least or at about 2 hour, after at least or at about 2.5 hours, after at least or at about 3 hours, after at least or at about 3.5 hours, or after at least or at about 4 hours, or any time between 15 min and 6 hours, optionally any time during said interval or other intervals such as between 75 min and 4.5 hours of EVLP.

During an EVLP assessment for example, the EVLP circuit can be primed with 2000 mL of STEEN Solution™ (or equivalent). Subsequently, the circulating STEEN Solution™ within the EVLP circuit can be replenished optionally in the following manner (for example): at the end of the first hour, one-half litre of the perfusate is removed from the circuit, and one-half litre of fresh STEEN Solution™ is replaced into the circuit. After this, at the end of each subsequent hour, 0 to 500 mL of perfusate fluid can be removed from the circuit, and 500 mL of fresh STEEN Solution™ can be added to the circuit. As described below, at the end of four hours of perfusion, aliquots of the test EVLP perfusate fluid were withdrawn from the perfusion circuit and frozen. The skilled person can readily determine the time and amount of perfusate fluid to be removed or replaced.

In an embodiment, the test EVLP perfusate sample is collected after at least or at about 15 min, at least or at about 30 min, at least or at about 45 min of EVLP, after at least or at about 1 hour of EVLP, after at least or at about 75 min of EVLP, after at least or at about 90 min of EVLP, after at least or at about 105 min of EVLP, after at least or at about 2 hours of EVLP, after at least or at about 3 hours of EVLP or after at least or at about 4 hours of EVLP. Test EVLP perfusate samples can also be collected at other times for example, after at least or at about 1.5 hours, after at least or at about 2.5 hours, after at least or at about 3.5 hours or after at least or at about 4.5 hours. Perfusate samples can also be collected for example at or after about 5 hours or 6 hours of EVLP. The test EVLP perfusate samples are collected within a time interval, with multiple collection within the time interval, without restriction on the intervening time between first, second and any subsequent collection of test EVLP perfusate samples. In some embodiments, the test EVLP perfusate sample is collected within a time interval, for example between any of the foregoing times. The skilled person can readily recognize that intervening time between any two perfusate sample collection can be regular or irregular, such that the intervening time between collecting any two test EVLP perfusate samples vary from another intervening time between collection any other two test EVLP perfusate samples. In some embodiments, intervening time between any two collection of EVLP perfusate samples is any time therebetween 1 min and 6 hours, optionally about 1, 2, 3, 4, 5, 6, 7, 8, 9, 10, 11, 12, 15, 20, 25, 30, 35, 40, 45, 50, 55, 60, 65, 70, 75, 80, 85, 90, 100, 105, 110, 120, 135, 150, 165, 180 min, optionally about 2, 2.5, 3, 3.5, 4, 4.5, 5, 5.5 or 6 hours. Optionally the perfusate sample is collected between 15 min and 4 hours of EVLP, between 1 hour and 3 hours of EVLP, between 1.5 hour and 3 hours of EVLP, between 1.5 hours and 2.5 hours of EVLP or between 1 hour and 2 hours of EVLP. Other ranges can also be considered including ranges between 45 min and 6 hours.

In an embodiment, more than one test EVLP perfusate sample is collected. For example, 2 or more test EVLP perfusate samples can be collected at regular or irregular intervals. Any interval of time (e.g. every 1 min, 5 min, 10 min or 15 min or longer such as 20 min, 30 min, 45 min or 1 hour) as convenient can be assessed.

In an embodiment, a first test EVLP perfusate sample is collected after at least or at about 45 min of EVLP, and one or more subsequent test EVLP perfusate samples are collected any time therebetween 1 min and 6 hours of collecting the first perfusate sample, optionally intervening time between collecting the first and the subsequent, or any two subsequent test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 105, 110, 120, 135, 150, 165, or 180 min, optionally about 2, 2.5, 3, 3.5, 4, 4.5, 5, 5.5 or 6 hours, optionally any time therebetween 1 min and 6 hours.

In an embodiment, the level or one or more parameter values is concentration of at least three of IL-8, IL-6, IL-10 and IL-1β optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-10, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-1β, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of sTNFR1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-8, and IL-10 optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-8 and IL-1β optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-6, IL-10, and IL-1β optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8, IL-10, and IL-1β optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion. In an embodiment, the level or one or more parameter values is concentration of IL-8, IL-6, IL-10 and IL-1β optionally in combination with one or both of sTNFR1 and sTREM1, wherein the one or more perfusate samples is a perfusate sample taken after at least or at about 45, 60, 75, 90, 105, 120, 135, 150, 165, 180, 195, 210, 225, 240, 255, 270, 285, 300, 315, 330, 345 or 360 min of perfusion, or any time therebetween 45 min and 360 min of perfusion.

One or more biochemical and/or physiological parameter values can be assed at any time after the start of EVLP, such as after at least or at about 15 min, at least or at about 30 min, at least or at about 45 min, after at least or at about 1 hour, after at least or at about 1.5 hours, after at least or at about 2 hour, after at least or at about 2.5 hours, after at least or at about 3 hours, after at least or at about 3.5 hours, or after at least or at about 4 hours, or any time between 15 min and 6 hours, optionally any time during said interval or other intervals such as between 75 min and 4.5 hours of EVLP or described herein for one or more biomarkers.

As demonstrated in the Examples, IL-6, IL-8, IL-10 and IL-1β, optionally in combination with one or both of sTNFR1 and sTREM1 were differentially detected and could be used to identify donor lungs that were associated with poor PO. An elevated product quotient for the biomarkers tested was associated with poorer outcome. For example, the level of IL-1β, IL-8, IL-6, IL-10, sTNFR1 and/or sTREM1, as well as the rate of IL-1β, IL-8, IL-10, IL-6, sTNFR1 and/or sTREM1 production during EVLP was on average increased in donor lungs that were transplanted to patients that exhibited prolonged ICU stays after transplant. Similarly, IL-1β, IL-8, IL-10, IL-6 sTNFR1 and/or sTREM1 levels measured at 4 hours of EVLP were found to be significantly increased in donors that experienced prolonged ICU stays.

In an embodiment, the level or one or more parameter values determined is rate of IL-8, IL-6, and IL-10 production optionally in combination with rate of one or both of sTNFR1 and sTREM1 production.

In an embodiment, the level or one or more parameter values determined is rate of IL-6, IL-8, and IL-1β production optionally in combination with rate of one or both of sTNFR1 and sTREM1 production.

In an embodiment, the level or one or more parameter values determined is rate of IL-6, IL-10, and IL-1β production optionally in combination with rate of one or both of sTNFR1 and sTREM1 production.

In an embodiment, the level or one or more parameter values determined is rate of IL-8, IL-10 and IL-1β production optionally in combination with rate of one or both of sTNFR1 and sTREM1 production.

In an embodiment, the level or one or more parameter values determined is rate of IL-6, IL-8, IL-10 and IL-1β production optionally in combination with rate of one or both of sTNFR1 and sTREM1 production.

In another embodiment, the level or one or more parameter values is concentration of IL-8, IL-6, and IL-10, optionally in combination with concentration of one or both of sTNFR1 and sTREM1, wherein the one or more perfusate samples is a perfusate sample taken after at least or at about a time described herein, optionally after at least or at about 1 hour or 75 min of perfusion.

In an embodiment, the level or one or more parameter values is concentration of IL-8, IL-6 and IL-10, optionally in combination with concentration of one or both of sTNFR1 and sTREM1, wherein the one or more perfusate samples is a perfusate sample taken at about 4 hours of perfusion.

In another embodiment, the level or one or more parameter values is concentration of IL-8, IL-6, and IL-1β, optionally in combination with concentration of one or both of sTNFR1 and sTREM1, wherein the one or more perfusate samples is a perfusate sample taken after at least or at about a time described herein, optionally after at least or at about 1 hour or 75 min of perfusion.

In an embodiment, the level or one or more parameter values is concentration of IL-8, IL-6 and IL-1β, optionally in combination with concentration of one or both of sTNFR1 and sTREM1, wherein the one or more perfusate samples is a perfusate sample taken at about 4 hours of perfusion.

In another embodiment, the level or one or more parameter values is concentration of IL-6, IL-10 and IL-1β, optionally in combination with concentration of one or both of sTNFR1 and sTREM1, wherein the one or more perfusate samples is a perfusate sample taken after at least or at about a time described herein, optionally after at least or at about 1 hour or 75 min of perfusion.

In an embodiment, the level or one or more parameter values is concentration of IL-6, IL-10 and IL-1β, optionally in combination with concentration of one or both of sTNFR1 and sTREM1, wherein the one or more perfusate samples is a perfusate sample taken at about 4 hours of perfusion.

In another embodiment, the level or one or more parameter values is concentration of IL-8, IL-10 and IL-1β, optionally in combination with concentration of one or both of sTNFR1 and sTREM1, wherein the one or more perfusate samples is a perfusate sample taken after at least or at about a time described herein, optionally after at least or at about 1 hour or 75 min of perfusion.

In an embodiment, the level or one or more parameter values is concentration of IL-8, IL-10 and IL-1β, optionally in combination with concentration of one or both of sTNFR1 and sTREM1, wherein the one or more perfusate samples is a perfusate sample taken at about 4 hours of perfusion.

In another embodiment, the level or one or more parameter values is concentration of IL-8, IL-6, IL-10 and IL-1β optionally in combination with concentration of one or both of sTNFR1 and sTREM1, wherein the one or more perfusate samples is a perfusate sample taken after at least or at about a time described herein, optionally after at least or at about 1 hour or 75 min of perfusion.

In an embodiment, the level or one or more parameter values is concentration of IL-8, IL-6, IL-10 and IL-1β, optionally in combination with concentration of one or both of sTNFR1 and sTREM1, wherein the one or more perfusate samples is a perfusate sample taken at about 4 hours of perfusion.

In an embodiment, the levels of IL-8, IL-6, and IL-10 polypeptides and optionally one or both of sTNFR1 and sTREM1, are indicative that the donor lung optionally DCD donor lung, optionally DBD donor lung, has an increased risk of poor PO, is less suitable or unsuitable for transplant or will be declined at the end of EVLP is an increase of at least 1.2×, 1.3×, 1.4×, 1.5×, 1.6×, 1.7×, 1.8×, 1.9×, or 2× and up to 5×, optionally any value therebetween 2× and 5×, compared to control, e.g. wherein the poor PO is >3 ICU days post-transplant. In an embodiment, the levels of IL-8, IL-6, and IL-10 polypeptides and optionally one or both of sTNFR1 and sTREM1, are indicative that the donor lung, optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >3 ICU days post-transplant.

In an embodiment, the levels of IL-8, IL-6, and IL-1β polypeptides and optionally one or both of sTNFR1 and sTREM1, are indicative that the donor lung, optionally DCD donor lung, optionally DBD donor lung, has an increased risk of poor PO is less or unsuitable for transplant or will be declined at the end of EVLP is an increase of at least 1.2×, 1.3×, 1.4×, 1.5×, 1.6×, 1.7×, 1.8×, 1.9×, or 2×, and up to 5×, optionally any value therebetween 2× and 5×, compared to control, e.g. wherein the poor PO is >3 ICU days post-transplant. In an embodiment, the levels of IL-8, IL-6, and IL-1β polypeptides and optionally one or both of sTNFR1 and sTREM1, are indicative that the donor lung, optionally DCD donor lung, optionally DBD donor lung has an increased risk of poor PO, is less or unsuitable for transplant or will be declined at the end of EVLP is an increase of any value therebetween 2× and 5×, compared to control, e.g. wherein the poor PO is >3 ICU days post-transplant.

In an embodiment, the levels of the IL-6, IL-10, and IL-1β polypeptides and optionally one or both of sTNFR1 and sTREM1, are indicative that the donor lung, optionally DCD donor lung, optionally DBD donor lung, has an increased risk of poor PO, is less or unsuitable for transplant or will be declined at the end of EVLP is an increase of at least 1.2×, 1.3×, 1.4×, 1.5×, 1.6×, 1.7×, 1.8×, 1.9×, or 2×, and up to 5×, optionally any value therebetween 2× and 5×, compared to control, e.g. poor PO is >3 ICU days post-transplant. In an embodiment, the levels of the IL-6, IL-10, and IL-1β polypeptides and optionally one or both of sTNFR1 and sTREM1, are indicative that the donor lung, optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >3 ICU days post-transplant.

In an embodiment, the levels of the IL-8, IL-10, and IL-1β polypeptides and optionally one or both of sTNFR1 and sTREM1, are indicative that the donor lung, optionally DCD donor lung, optionally DBD donor lung, has an increased risk of poor PO, is less or unsuitable for transplant or will be declined at the end of EVLP is an increase of at least 1.2×, 1.3×, 1.4×, 1.5×, 1.6×, 1.7×, 1.8×, 1.9×, or 2×, and up to 5×, compared to control, e.g. poor PO is >3 ICU days post-transplant. In an embodiment, the levels of the IL-8, IL-10, and IL-1β polypeptides and optionally one or both of sTNFR1 and sTREM1, are indicative that the donor lung optionally DCD donor lung, optionally DBD donor lung, has an increased risk of poor PO, is less or unsuitable for transplant or will be declined at the end of EVLP is an increase of any value therebetween 2× and 5×, compared to control, e.g. wherein the poor PO is >3 ICU days post-transplant.

In one embodiment, the combination of at least three biomarkers and/or the one or more parameters are parameters selected from any of the foregoing: 1) IL-6 concentration after at least or at about 1 hour of perfusion and about 4 hours of perfusion or any time therebetween, optionally between 1 hour and 3 hours or between 1.5 hours and 2.5 hours of perfusion; 2) IL-8 concentration after at least or at about 1 hour of perfusion and about 4 hours of perfusion or any time therebetween, optionally between 1 hour and 3 hours or between 1.5 hours and 2.5 hours of perfusion; 3) IL-10 concentration after at least or at about 1 hour of perfusion and about 4 hours of perfusion or any time therebetween, optionally between 1 hour and 3 hours or between 1.5 hours and 2.5 hours of perfusion; 4) IL-1β concentration after at least or at about 1 hour of perfusion and about 4 hours of perfusion or any time therebetween, optionally between 1 hour and 3 hours or between 1.5 hours and 2.5 hours of perfusion; 5) IL-6 and IL-8 concentrations after at least or at about 1 hour of perfusion and about 4 hours of perfusion or any time therebetween, optionally between 1 hour and 3 hours or between 1.5 hours and 2.5 hours of perfusion; 6) IL-6 and IL-10 concentrations after at least or at about 1 hour of perfusion and about 4 hours of perfusion or any time therebetween, optionally between 1 hour and 3 hours or between 1.5 hours and 2.5 hours of perfusion; 7) IL-6 and IL-1β concentrations after at least or at about 1 hour of perfusion and about 4 hours of perfusion or any time therebetween, optionally between 1 hour and 3 hours or between 1.5 hours and 2.5 hours of perfusion; 8) IL-8 and IL-10 after at least or at about 1 hour of perfusion and about 4 hours of perfusion or any time therebetween, optionally between 1 hour and 3 hours or between 1.5 hours and 2.5 hours of perfusion; 9) IL-8 and IL-1β concentrations after at least or at about 1 hour of perfusion and about 4 hours of perfusion or any time therebetween, optionally between 1 hour and 3 hours or between 1.5 hours and 2.5 hours of perfusion; 10) IL-10 and IL-1β concentrations after at least or at about 1 hour of perfusion and about 4 hours of perfusion or any time therebetween, optionally between 1 hour and 3 hours or between 1.5 hours and 2.5 hours of perfusion; 11) IL-6, IL-8 and IL-10 concentrations after at least or at about 1 hour of perfusion and about 4 hours of perfusion or any time therebetween, optionally between 1 hour and 3 hours or between 1.5 hours and 2.5 hours of perfusion; 12) IL-6, IL-8, and IL-1β concentrations after at least or at about 1 hour of perfusion and about 4 hours of perfusion or any time therebetween, optionally between 1 hour and 3 hours or between 1.5 hours and 2.5 hours of perfusion 13) IL-6, IL-10, and IL-1β concentrations after at least or at about 1 hour of perfusion and about 4 hours of perfusion or any time therebetween, optionally between 1 hour and 3 hours or between 1.5 hours and 2.5 hours of perfusion; 14) IL-8, IL-10, and IL-1β concentrations after at least or at about 1 hour of perfusion and about 4 hours of perfusion or any time therebetween, optionally between 1 hour and 3 hours or between 1.5 hours and 2.5 hours of perfusion; and 15) IL-6, IL-8, IL-10 and IL-1β concentrations after at least or at about 1 hour of perfusion and about 4 hours of perfusion or any time therebetween, optionally between 1 hour and 3 hours or between 1.5 hours and 2.5 hours of perfusion. In some embodiments, the combination of at least three biomarkers and/or the one or more parameters further comprises biomarkers and/or parameters selected from the concentration of one or both of sTNFR1 and sTREM1, after at least or at about 1 hour of perfusion and about 4 hours of perfusion or any time therebetween, optionally between 1 hour and 3 hours or between 1.5 hours and 2.5 hours of perfusion.

Accordingly in an embodiment, the combinations include at least three biomarkers of one or more of A) IL-6; IL-8; IL-10; IL-1β; IL-6 and IL-8; IL-10 and IL-1β; IL-6 and IL-10; IL-6 and IL-1β; IL-8 and IL-10; IL-8 and IL-1β; IL-6, IL-8 and IL-10; IL-6, IL-8 and IL-1β; IL-8, IL-10 and IL-1β; IL-6, IL-10, and IL-1β; and IL-6, IL-8, IL-10 and IL-1β; any of the foregoing optionally further comprising sTNFR1 and sTREM1, combining with one or more of EVLP perfusate sample collected at B) any time therebetween 45 min and 6 hours of EVLP, optionally after at least or at about 45 min of EVLP, after at least or at about 1 hour of EVLP, after at least or at about 75 min of EVLP, after at least or at about 1.5 hours of EVLP, after at least or at about 105 min of EVLP, after at least or at about 2 hours of EVLP, after at least or at about 2.5 hours of EVLP, after at least or at about 3 hours of EVLP, after at least or at about 3.5 hours of EVLP, after at least or at about 4 hours of EVLP, after at least or at about 4.5 hours of EVLP, after at least or at about 5 hours of EVLP, after at least or at about 5.5 hours of EVLP, after at least or at about 6 hours of EVLP, optionally intervening time between collecting any two test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 110, 120, 150 min, optionally about 3, 3.5, 4, 4.5, 5, 5.5 or 6 hours, optionally any time therebetween 1 min and 6 hours.

In an embodiment, the parameters are at least one of the combination selected from: 1) IL-8 concentration after at least or at about 4 hours of perfusion, IL-6 concentration after at least or at about 4 hours of perfusion, and IL-10 concentration after at least or at about 4 hours of perfusion; 2) IL-8 concentration after at least or at about 4 hours of perfusion, IL-6 concentration after at least or at about 4 hours of perfusion, and IL-1β concentration after at least or at about 4 hours of perfusion; 3) IL-1β concentration after at least or at about 4 hours of perfusion, IL-6 concentration after at least or at about 4 hours of perfusion, and IL-10 concentration after at least or at about 4 hours of perfusion; 4) IL-1β concentration after at least or at about 4 hours of perfusion, IL-8 concentration after at least or at about 4 hours of perfusion, and IL-10 concentration after at least or at about 4 hours of perfusion; 5) IL-6 concentration after at least or at about 1 hour of perfusion, IL-8 concentration after at least or at about 4 hours of perfusion, and IL-10 concentration after at least or at about 4 hours of perfusion; 6) IL-6 concentration after at least or at about 4 hours of perfusion, IL-8 concentration after at least or at about 1 hour of perfusion, and IL-10 concentration after at least or at about 4 hours of perfusion; 7) IL-6 concentration after at least or at about 4 hours of perfusion, IL-8 concentration after at least or at about 4 hours of perfusion, and IL-10 concentration after at least or at about 1 hour of perfusion; 8) IL-6 concentration after at least or at about 1 hour of perfusion, IL-8 concentration after at least or at about 1 hour of perfusion, and IL-10 concentration after at least or at about 4 hours of perfusion; 9) IL-6 concentration after at least or at about 1 hour of perfusion, IL-8 concentration after at least or at about 4 hours of perfusion, and IL-10 concentration after at least or at about 1 hour of perfusion; 10) IL-6 concentration after at least or at about 4 hours of perfusion, IL-8 concentration after at least or at about 1 hour of perfusion, and IL-10 concentration after at least or at about 1 hour of perfusion; 11) IL-6 concentration after at least or at about 1 hour of perfusion, IL-8 concentration after at least or at about 1 hour of perfusion, and IL-10 concentration after at least or at about 1 hour of perfusion; 12) IL-6 concentration after at least or at about 1 hour of perfusion, IL-8 concentration after at least or at about 4 hours of perfusion, and IL-1β concentration after at least or at about 4 hours of perfusion; 13) IL-6 concentration after at least or at about 4 hours of perfusion, IL-8 concentration after at least or at about 1 hour of perfusion, and IL-1β concentration after at least or at about 4 hours of perfusion; 14) IL-6 concentration after at least or at about 4 hours of perfusion, IL-8 concentration after at least or at about 4 hours of perfusion, and IL-1β concentration after at least or at about 1 hour of perfusion; 15) IL-6 concentration after at least or at about 1 hour of perfusion, IL-8 concentration after at least or at about 1 hour of perfusion, and IL-1β concentration after at least or at about 4 hours of perfusion; 16) IL-6 concentration after at least or at about 1 hour of perfusion, IL-8 concentration after at least or at about 4 hours of perfusion, and IL-1β concentration after at least or at about 1 hour of perfusion; 17) IL-6 concentration after at least or at about 4 hours of perfusion, IL-8 concentration after at least or at about 1 hour of perfusion, and IL-1β concentration after at least or at about 1 hour of perfusion; 18) IL-6 concentration after at least or at about 1 hour of perfusion, IL-8 concentration after at least or at about 1 hour of perfusion, and IL-1β concentration after at least or at about 1 hour of perfusion; 19) IL-6 concentration after at least or at about 1 hour of perfusion, IL-10 concentration after at least or at about 4 hours of perfusion, and IL-1β concentration after at least or at about 4 hours of perfusion; 20) IL-6 concentration after at least or at about 4 hours of perfusion, IL-10 concentration after at least or at about 1 hour of perfusion, and IL-1β concentration after at least or at about 4 hours of perfusion; 21) IL-6 concentration after at least or at about 4 hours of perfusion, IL-10 concentration after at least or at about 4 hours of perfusion, and IL-1β concentration after at least or at about 1 hour of perfusion; 22) IL-6 concentration after at least or at about 1 hour of perfusion, IL-10 concentration after at least or at about 1 hour of perfusion, and IL-1β concentration after at least or at about 4 hours of perfusion; 23) IL-6 concentration after at least or at about 1 hour of perfusion, IL-10 concentration after at least or at about 4 hours of perfusion, and IL-1β concentration after at least or at about 1 hour of perfusion; 24) IL-6 concentration after at least or at about 4 hours of perfusion, IL-10 concentration after at least or at about 1 hour of perfusion, and IL-1β concentration after at least or at about 1 hour of perfusion; 25) IL-6 concentration after at least or at about 1 hour of perfusion, IL-10 concentration after at least or at about 1 hour of perfusion, and IL-1β concentration after at least or at about 1 hour of perfusion; 26) IL-10 concentration after at least or at about 1 hour of perfusion, IL-8 concentration after at least or at about 4 hours of perfusion, and IL-1β concentration after at least or at about 4 hours of perfusion; 27) IL-10 concentration after at least or at about 4 hours of perfusion, IL-8 concentration after at least or at about 1 hour of perfusion, and IL-1β concentration after at least or at about 4 hours of perfusion; 28) IL-10 concentration after at least or at about 4 hours of perfusion, IL-8 concentration after at least or at about 4 hours of perfusion, and IL-1β concentration after at least or at about 1 hour of perfusion; 29) IL-10 concentration after at least or at about 1 hour of perfusion, IL-8 concentration after at least or at about 1 hour of perfusion, and IL-1β concentration after at least or at about 4 hours of perfusion; 30) IL-10 concentration after at least or at about 1 hour of perfusion, IL-8 concentration after at least or at about 4 hours of perfusion, and IL-1β concentration after at least or at about 1 hour of perfusion; 31) IL-10 concentration after at least or at about 4 hours of perfusion, IL-8 concentration after at least or at about 1 hour of perfusion, and IL-1β concentration after at least or at about 1 hour of perfusion; 32) IL-10 concentration after at least or at about 1 hour of perfusion, IL-8 concentration after at least or at about 1 hour of perfusion, and IL-1β concentration after at least or at about 1 hour of perfusion; 33) IL-6 concentration after at least or at about 4 hours of perfusion, IL-8 concentration after at least or at about 4 hours of perfusion, IL-10 concentration after at least or at about 4 hours of perfusion and IL-1β concentration after at least or at about 4 hours of perfusion; 34) IL-6 concentration after at least or at about 1 hour of perfusion, IL-8 concentration after at least or at about 4 hours of perfusion, IL-10 concentration after at least or at about 4 hours of perfusion and IL-1β concentration after at least or at about 4 hours of perfusion; 35) IL-6 concentration after at least or at about 4 hours of perfusion, IL-8 concentration after at least or at about 1 hour of perfusion, IL-10 concentration after at least or at about 4 hours of perfusion and IL-1β concentration after at least or at about 4 hours of perfusion; 36) IL-6 concentration after at least or at about 4 hours of perfusion, IL-8 concentration after at least or at about 4 hours of perfusion, IL-10 concentration after at least or at about 1 hour of perfusion and IL-1β concentration after at least or at about 4 hours of perfusion; 37) IL-6 concentration after at least or at about 4 hours of perfusion, IL-8 concentration after at least or at about 4 hours of perfusion, IL-10 concentration after at least or at about 4 hours of perfusion and IL-1β concentration after at least or at about 1 hour of perfusion; 38) IL-6 concentration after at least or at about 1 hour of perfusion, IL-8 concentration after at least or at about 1 hour of perfusion, IL-10 concentration after at least or at about 4 hours of perfusion and IL-1β concentration after at least or at about 4 hours of perfusion; 39) IL-6 concentration after at least or at about 1 hour of perfusion, IL-8 concentration after at least or at about 4 hours of perfusion, IL-10 concentration after at least or at about 1 hour of perfusion and IL-1β concentration after at least or at about 4 hours of perfusion; 40) IL-6 concentration after at least or at about 1 hour of perfusion, IL-8 concentration after at least or at about 4 hours of perfusion, IL-10 concentration after at least or at about 4 hours of perfusion and IL-1β concentration after at least or at about 1 hour of perfusion; 41) IL-6 concentration after at least or at about 4 hours of perfusion, IL-8 concentration after at least or at about 1 hour of perfusion, IL-10 concentration after at least or at about 1 hour of perfusion and IL-1β concentration after at least or at about 4 hours of perfusion; 42) IL-6 concentration after at least or at about 4 hours of perfusion, IL-8 concentration after at least or at about 1 hour of perfusion, IL-10 concentration after at least or at about 4 hours of perfusion and IL-1β concentration after at least or at about 1 hour of perfusion; 43) IL-6 concentration after at least or at about 4 hours of perfusion, IL-8 concentration after at least or at about 4 hours of perfusion, IL-10 concentration after at least or at about 1 hour of perfusion and IL-1β concentration after at least or at about 1 hour of perfusion; 44) IL-6 concentration after at least or at about 1 hour of perfusion, IL-8 concentration after at least or at about 1 hour of perfusion, IL-10 concentration after at least or at about 1 hour of perfusion and IL-1β concentration after at least or at about 4 hours of perfusion; 45) IL-6 concentration after at least or at about 1 hour of perfusion, IL-8 concentration after at least or at about 1 hour of perfusion, IL-10 concentration after at least or at about 4 hours of perfusion and IL-1β concentration after at least or at about 1 hour of perfusion; 46) IL-6 concentration after at least or at about 1 hour of perfusion, IL-8 concentration after at least or at about 4 hours of perfusion, IL-10 concentration after at least or at about 1 hour of perfusion and IL-1β concentration after at least or at about 1 hour of perfusion; 47) IL-6 concentration after at least or at about 4 hours of perfusion, IL-8 concentration after at least or at about 1 hour of perfusion, IL-10 concentration after at least or at about 1 hour of perfusion and IL-1β concentration after at least or at about 1 hour of perfusion; 48) IL-6 concentration after at least or at about 1 hour of perfusion, IL-8 concentration after at least or at about 1 hour of perfusion, IL-10 concentration after at least or at about 1 hour of perfusion and IL-1β concentration after at least or at about 1 hour of perfusion. In some embodiments, the one or more parameters further comprises parameters selected from the concentration (e.g. level) of one or both of sTNFR1 and sTREM1.

One or more biochemical and/or physiological parameter values can be assed at any time after the start of EVLP, along with any of the combinations described herein.

In an embodiment, the one or more parameter values related to a level of three or more of IL-8, IL-6, IL-10 and IL-1β and optionally one or both of sTNFR1 and sTREM1 polypeptides are detected for example using a bead based proteomic assay such as the Luminex® assay.

In an embodiment, the one or more parameter values related to a level one or more IL-8, IL-6, IL-10 and IL-1β and optionally one or both of sTNFR1 and sTREM1 polypeptide is detected or determined by immunohistochemistry (IHC), or immunoassays including multiplex assays such as Luminex® based assays, Western blots, ELISA, immunofluorescence, radioimmunoassay, dot blotting, flow cytometry, FACS, protein microarray, immunoprecipitation followed by SDS-PAGE, immunocytochemistry, or mass spectrometry. For example, the one or more parameter values related to a level of a biomarker of the invention can be measured using immunoassay platforms such as a Simple Plex (Protein Simple, San Jose, Calif., USA). In some embodiments, the perfusate is diluted prior to any measuring assays. Any assay capable of determining a biomarker concentration can be used.

In an embodiment, the one or more parameter values related to a level are determined by ELISA.

An at least 1.2 fold difference means for example that the level of the biomarkers in the test perfusate sample is at least 120% the level in a control comparator perfusate sample or derived value.

As mentioned above, the comparing step or steps can be performed for example, by interrogating a database comprising the cut-off level or reference score. For example, comparing the one or more parameter values related to a level of each the at least three biomarkers in the perfusate sample with a cut-off level or reference score, for example when also assessing one or more biochemical and physiological parameters can be performed by querying a database. The can be a series of values or a range comprising or determined from known concentrations/values that are associated with transplant outcome (ex. PGD status, ICU LOS) to arrive at a ranking/injury score.

For example, measured biomarker levels can be compared to a database of previously determined levels and/or parameters of known patient outcome/suitability for transplant determinations to identify the outcomes with most similar levels, thereby predicting risk or likelihood for example, the likely patient outcome or suitability for transplant of a particular organ. The cut-off value and/or reference score can also be a range determined from levels and/or parameter values of similar patient outcome patients and/or transplant suitability outcomes. If a test case falls within a range, it is predicted for example to have a similar risk or likelihood such as outcome or suitability for transplant, or likelihood a donor lung will be declined at the end of the EVLP process.

In an embodiment, the method involves comparing to a cut-off or reference score. For example, each marker will have a different cut-off depending on statistical calculations and/or desired test sensitivity and/or specificity. Where more than one biomarker and optionally one or more biochemical and/or physiological parameters is assessed, a composite score, e.g. a reference score, can be determined.

In an embodiment, the poor outcome lung grafts are characterized as being unsuitable for clinical transplantation after EVLP or, in the recipient after transplantation, inducing death from graft-related causes within 30 days, PGD3 or requiring extracorporeal life support/ECMO, or prolonged hospital/ICU/mechanical ventilation days (e.g. greater than 3 days ICU and/or mechanical ventilation).

As demonstrated in the Examples, the clinical measures of patient outcome (e.g. ICU length of stay etc.) were found to be significantly correlated with each other; this correlation between measures of outcome was also found to exist for a patient's APACHE score. For example, the correlation between ICU LOS and APACHE score was found to be 0.35 (p<0.0001).

Embodiments of systems and methods disclosed in present subject matter may be implemented as separate modules, as a single module, or any combination thereof. The embodiments may be implemented using any software development environment or computer language. The embodiments may be provided as a packaged software product, a web-service, an API or any other means of software service.

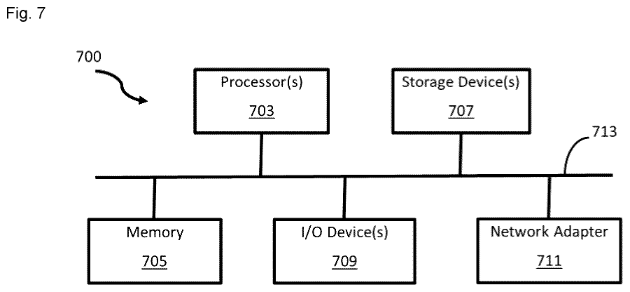

FIG. 7 is a block diagram of a computer system 700 as may be used to implement features of the disclosed embodiments in the present subject matter. The computing system 700 may be used to implement any of the entities, methods, components or services depicted in the present subject matter. The computing system 700 may include one or more central processing units (“processors”) 703, memory 705, input/output devices 709 (e.g., keyboard and pointing devices, display devices), storage devices 707 (e.g., disk drives), and network adapters 611 (e.g., network interfaces) that are connected to an interconnect 713. The interconnect 713 may represent any one or more separate physical buses, point to point connections, or both connected by appropriate bridges, adapters, or controllers. The interconnect 713, therefore, may include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an Institute of Electrical and Electronics Components (IEEE) standard 1394 bus, also called “Firewire”.

The memory 705 and storage devices 707 are computer-readable storage media that may store instructions that implement at least portions of the described embodiments. For example, the memory may include instructions that when executed by a processing unit, cause the processing unit to implement the methods disclosed in the present subject matter. In addition, the data structures and message structures may be stored or transmitted via a data transmission medium, such as a signal on a communications link. Various communications links may be used, such as the Internet, a local area network, a wide area network, or a point-to-point dial-up connection. Thus, computer readable media can include computer-readable storage media (e.g., “non-transitory” media) and computer-readable transmission media.

The instructions stored in memory 605 can be implemented as software and/or firmware to program the processor(s) 603 to carry out actions described above. In some embodiments, such software or firmware may be initially provided to the system 600 by downloading it from a remote system through the system 600 (e.g., via network adapter 611).

The embodiments introduced herein can be implemented by, for example, programmable circuitry (e.g., one or more microprocessors) programmed with software and/or firmware, or entirely in special-purpose hardwired (non-programmable) circuitry, or in a combination of such forms. Special-purpose hardwired circuitry may be in the form of, for example, one or more ASICs, PLDs, FPGAs, etc.

The embodiments disclosed above may be implemented as separate modules, e.g., as presented above, as a single module, or any combination thereof. Implementation details may vary, including machine learning algorithms employed. The embodiments may be implemented using any software development environment or computer language. The embodiments may be provided as a packaged software product, a web-service, an API or any other means of software service.

The computer-implemented method for the detecting for example of outcome/risk, suitability for transplant, likelihood of decline at the end of EVLP or other methods described herein as it relates to donor lungs in a recipient post-transplant can employ the use of a processor/computer system as disclosed in the present subject matter. For example, a computer system comprising a processor is coupled to a memory storing computer program code to implement the methods described in the present subject matter. The computer is also coupled to memory and to interfaces such as a computer screen, keyboard, mouse, and printer, as well as other interfaces, such as a network interface, and software interfaces including a database interface. The computer may accept user input from a data input device, such as a keyboard, input data file, or network interface, or another system. The computer may provide an output to an output device such as a printer, display, network interface, or data storage device. An input device, for example a network interface, may receive an input (e.g. data, more specifically EVLP data) according to the present subject. The output device may provide an output (e.g. a display, including one or more numbers, a graph; a score, etc.).

In some embodiments, a computing device comprises a processor; and a system memory connected to the processor, the system memory including instructions that, when executed by the processor, cause the processor to obtain data (e.g. one or more parameter values) including for example EVLP data relating to one or more test EVLP perfusate samples of a donor lung. For example, the EVLP data may include one or more parameter values relating to a level of polypeptide of at least three biomarkers selected from IL-6, IL-8, IL-10, and IL-1β, optionally in combination with one or both of sTNFR1 and sTREM1. For example, a user may enter the EVLP data into a computer system via a user interface as disclosed herein.

For example, the processor may query a database of control EVLP data (e.g. known concentration data) or a cut-off level associated with patient outcome or transplant suitability to compare the EVLP data to the control EVLP data or cut-off level. The processor may determine a ranking score based on the comparison between the EVLP data and the control EVLP data (e.g. known concentration data) or the cut-off level.

The processor may combine the EVLP data with biochemical data and/or the physiological data. The processor may analyze the combined data to extract multiple features indicating characteristics of the donor lung and/or a likely patient outcome or suitability for transplant etc. Extracting the multiple features may include extracting the multiple features using an artificial intelligence technique or a machine learning technique. The processor may generate multiple classifiers/scores based on the multiple features. Generating the multiple classifiers/scores may include generating the multiple classifiers using the artificial intelligence technique or the machine learning technique. The machine learning technique may include a neural network. Each of the multiple classifiers/scores may indicate one of multiple outcomes. For example, the outcome may be related to a medical lung procedure using a donor lung, which was subject to EVLP.

A computer-program product is further described herein. The computer-program product can be used in conjunction with an electronic device. The computer-program product can include a non-transitory computer-readable storage medium and/or a computer-program mechanism embedded therein.

The computer-program product can include instructions for obtaining EVLP data relating to one or more test EVLP perfusate samples of a donor lung. The EVLP data can include one or more parameter values relating to a level of polypeptide of at least three biomarkers selected from IL-6, IL-8, IL-10, and IL-1β, optionally in combination with one or both of sTNFR1 and sTREM1.

The computer-program mechanism can also include: instructions for querying a database of control EVLP data or a cut-off level associated with patient outcome or transplant suitability to compare the EVLP data to the control EVLP data or cut-off level; and/or instructions for determining a ranking score based on the comparison between the EVLP data and the control EVLP data or cut-off level.

The computer-program product can further include: instructions for building a base model using biochemical data and/or the physiological data to determine the ranking score or combining the EVLP data to biochemical data and/or the physiological data prior to determining the ranking score; instructions for analyzing the combined data to extract multiple features indicating characteristics of the donor lung; and/or instructions for generating multiple classifiers/scores based on the multiple features, wherein each of the multiple classifiers/scores indicates one of multiple outcomes.

The computer-program product can further include: instructions for extracting the multiple features using an artificial intelligence technique or a machine learning technique; and/or instructions for generating the multiple classifiers using the artificial intelligence technique or the machine learning technique.

In an embodiment, the computer-program product may be packaged in software. For example, the computer program product may be available (e.g. for sale, testing, etc.) on the Internet through an online platform (such as a university or hospital website). For example, the computer program product may be available for sale through an online commerce platform.

When in use, the computer program product can allow a user to query a database of control EVLP data or cult-off levels. The database of control EVLP data (e.g. known concentration data) or the cut-off level can be associated with patient outcome or transplant suitability as described in the present subject matter.

For example, the user may query a database of control EVLP data or a cut-off level associated with patient outcome or transplant suitability to compare EVLP data to the control EVLP data or cut-off level. For example, the EVLP data may be obtained from test EVLP perfusate samples of a donor lung. The EVLP data can include one or more parameter values relating to a level of polypeptide of at least three biomarkers selected from IL-6, IL-8, IL-10, and IL-1β, optionally in combination with one or both of sTNFR1 and sTREM1.

The computer program product can allow the user to determine a ranking score based on the comparison between the EVLP data and the control EVLP data or the cut-off level.

The computer program product can also allow the user to build a base model using biochemical data and/or the physiological data. For example, this can be useful to determine a ranking score.

The computer program product can also allow the user to combine the EVLP data to biochemical data and/or the physiological data prior to determining the ranking score.

The computer program product can also allow the user to analyze the combined data to extract multiple features indicating characteristics of the donor lung.

The computer program product can allow the user to generate multiple classifiers/scores based on the multiple features, wherein each of the multiple classifiers/scores indicates one of multiple outcomes. The computer program product can also allow the user to extract the multiple features using an artificial intelligence technique or a machine learning technique. The computer program product can further allow the user to generate the multiple classifiers using the artificial intelligence technique or the machine learning technique.

The computer program product can be downloaded into a storage system, which can be memory storage of a remote computer, a laptop, a mobile phone, a network router, a switch, a bridge or a virtual space in a cloud environment, connected to an online commerce platform. The online commerce platform can enable a user to test the computer program product. The online commerce platform can display the computer program product characteristics and its selling price. The user can select and buy the computer program product from the online commerce platform using an appropriate payment method.

As disclosed herein, the biochemical data may include at least one of: base excess, bicarbonate, potassium, sodium, calcium, chloride, glucose, lactate and pH. The physiological data may include at least one of: driving pressure, PCO₂ (measured and differential), PO₂ (measured and differential), airway pressure, static and dynamic compliance, PA and/or LA pressure, and/or pulmonary vascular resistance.

Any combination of biochemical and/or physiologic parameter values may be used. In some embodiments, the combination comprises any two or more of calcium, glucose, lactate, pH, P02, and static and dynamic compliance.

Different combinations (biomarker plus one or more biochemical and physiologic parameter values to predict different outcomes and risks.

### III. Immunoassays and Kits

An aspect of the disclosure also includes kits containing antibodies for the detection of IL-6, IL-8, IL-10 and IL-1β, optionally in combination with one or both of sTNFR1 and sTREM1 that are used to measure the biomarker levels, i.e. polypeptide levels.

In an embodiment the kit comprises an immunoassay for one or more of biomarkers of the invention. Each kit comprises at least one detection antibody specific for a biomarker of the invention. For example, the antibody may be in the form of antibody coupled beads such as antibody coupled magnetic beads, or labelled antibodies, optionally comprised in a cartridge. In an embodiment, the kit further comprises one or more of a 96-well plate, a cartridge comprising one or more antibodies, standards, assay buffer, wash buffer, sample diluent, standard diluent, detection antibody diluent, streptavidin-PE, a filter plate and sealing tape. In an embodiment the kit comprises detection antibodies or assays for detecting three or more biomarkers of the invention e.g. IL-8, IL-6, IL-10 and IL-1β and optionally one or both of sTNFR1 and sTREM1. In an embodiment, the kit comprises detection antibodies or assays for detecting IL-6, IL-8, and IL-10 and optionally one or both of sTNFR1 and sTREM1. In another embodiment, the kit comprises detection antibodies or assays for detecting any one of IL-8 and IL-6 and one or more of IL-10 and IL-1β are assessed, optionally further comprising detection antibodies or assays for detecting one or both of sTNFR1 and sTREM1. In another embodiment, the kit comprises detection antibodies or assays for detecting IL-8 and at least two or three IL-6, IL-10 and IL-1β and optionally detection antibodies or assays for detecting one or both of sTNFR1 and sTREM1. In another embodiment, the kit comprises detection antibodies or assays for detecting IL-6 and at least two or three IL-8, IL-10 and IL-1β and optionally detection antibodies or assays for detecting one or both of sTNFR1 and sTREM1. In another embodiment, the kit comprises detection antibodies or assays for detecting IL-6, IL-1β and IL-10 and optionally detection antibodies or assays for detecting one or both of sTNFR1 and sTREM1. In another embodiment, the kit comprises detection antibodies or assays for detecting IL-8, IL-10 and IL-1β and optionally detection antibodies or assays for detecting one or both of sTNFR1 and sTREM1. In another embodiment, the kit comprises detection antibodies or assays for detecting IL-6, IL-8, IL-10, and IL-1β and optionally detection antibodies or assays for detecting one or both of sTNFR1 and sTREM1.

In an embodiment, the kits are for use for a method described herein.

In an embodiment, the kit further comprises detection agents for other known lung graft outcome markers.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

EVLP perfusate samples were collected during the course of an EVLP procedure. These perfusate samples were then analyzed for the polypeptide levels of IL-6, IL-8, IL-10, and IL-1β. The polypeptide levels (either absolute concentration, rate of increase or fold-increase) were included in a mathematical calculation that may include other known clinical variables (e.g. donor type). The output of this calculation provides a predictive score of whether or not the lung is likely to be declined at the end of EVLP and therefore it is prudent to stop EVLP at that time or, if transplanted, the patient that receives the lung is likely to experience a prolonged ICU stay. At that point the surgeon can then decide the relative risk to the patient and decide whether or not to carry out the transplant.

### Methods

Patient Selection: Approximately 60 clinical cases of lung transplantation involving EVLP were identified.

Perfusate Sample: Approximately 0.5 mL of Perfusate solution was removed from the EVLP circuit every 15 minutes during the course of the procedure (up to 6 hours), collected typically from 15 minutes to 4 hours. Perfusate samples were measured directly or snap frozen and used in the following experiments.

Biomarker Quantification: Perfusate samples were diluted as per manufacturer's instructions in calibrator diluent. A set of standards for the generation of a standard curve was prepared concurrently (Protein Simple). Each plate was prepared according to the manufacturer's protocol (Protein Simple). Each plate was then run and read on the Simple Plex System (Protein Simple), which was set up and calibrated as per the manufacturer's instructions. Levels of various markers including IL-6, IL-8, IL-10, and IL1β, were quantified.

Scoring Metric: A score was calculated by taking the product of the logarithmic concentration of each biomarker level measures. Version 1 (v1) is the product of the logarithmic concentration of IL-6 and IL-8. Version 2 (v2) is the product of the logarithmic concentration of IL-6, IL-8, IL-10 and IL1β.

Statistical Analysis: Analysis was carried out using Prism 7 (GraphPad), SPSS Statistics (IBM), or R software environment. For all statistical calculations, a p-value of less than 0.05 was considered statistically significant.

### Results

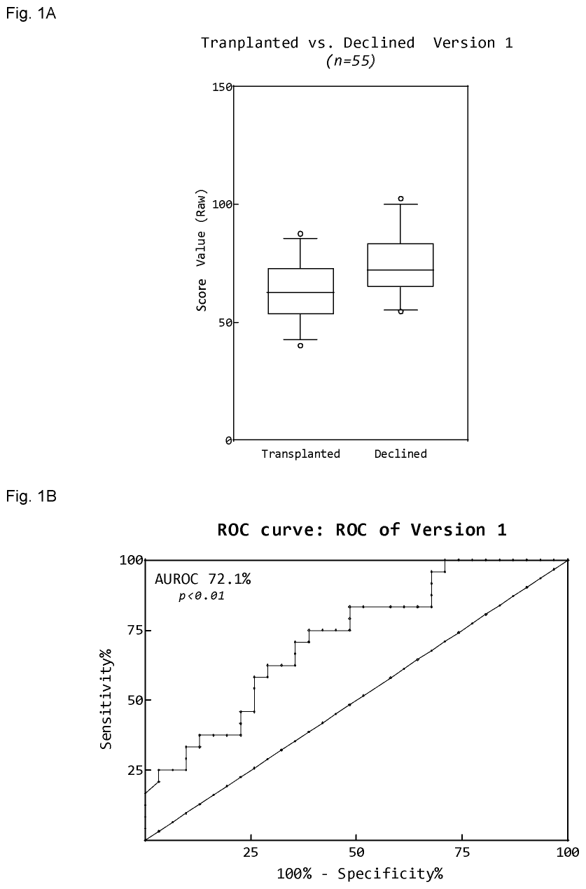

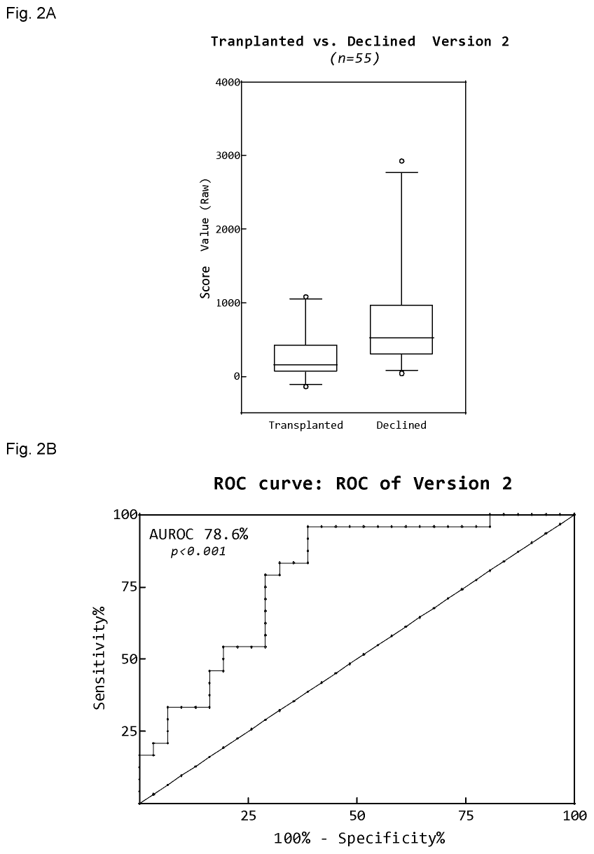

As shown in FIG. 1A, a scoring metric using biomarker concentrations of IL-6 and IL-8 show that there is a positive relation between the scores obtained using this combination of biomarkers (IL-6, and IL-8) and the EVLP outcome (transplanted vs. declined). The performance of this combination of biomarkers is shown in the ROC curve and the AUROC (area under ROC) (FIG. 1B). FIG. 2A and 2B show a more significant relationship between the scores obtained using the combination of biomarkers IL-6, IL-8, IL-10 and IL-1β and the EVLP outcomes. The performance of the combination of biomarkers IL-6, IL-8, IL-10 and IL-1β is shown in FIG. 2B.

Table 1 below shows the correlation between each biomarker and the recipient outcome such as ICU length of stay (LOS), hospital LOS and the APACHE Score. The correlation coefficient (r) is shown for the relationship between each biological mediator and a potential recipient outcome (n=21). The top 2 predictive mediators in each category are highlighted in bold.

TABLE 1
Correlation Coefficient between Biomarkers
and Recipient Outcome Indicators

| | Correlation Coefficient (r) | | |
|---|---|---|---|
| Biological Marker | ICU LOS | Hospital LOS | APACHE Score |
| IL-6 | 0.53 | 0.14 | 0.19 |
| IL-8 | 0.37 | 0.05 | 0.51 |
| IL-10 | 0.60 | 0.20 | 0.36 |
| IL-1β | 0.59 | −0.08 | 0.49 |

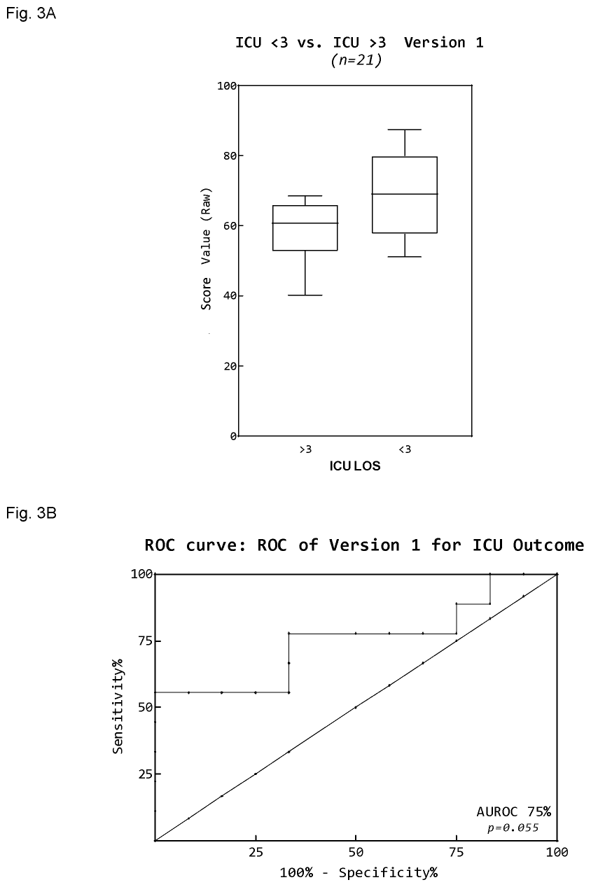

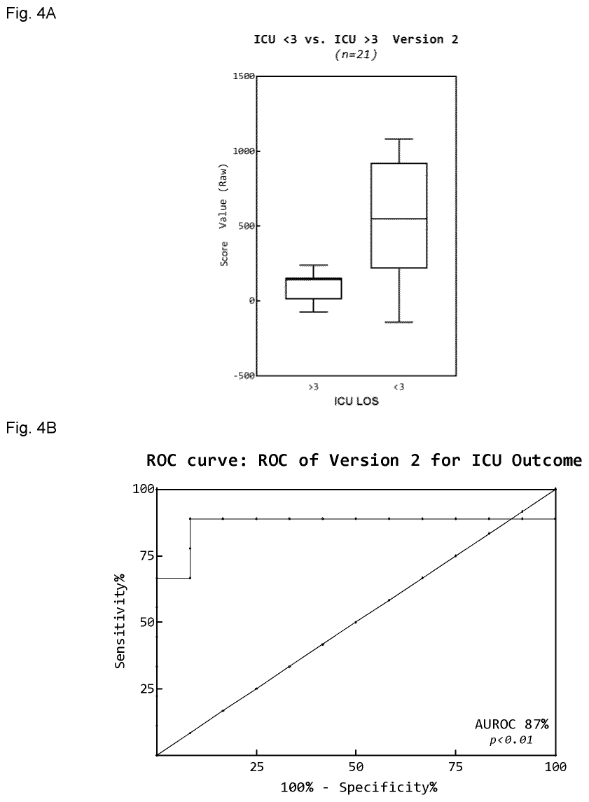

As shown in FIG. 3, there is a relationship between the scores obtained using the combination of biomarkers (IL-6, and IL-8) and the length of ICU stay (ICU LOS). A more significant relationship is observed for the combination of biomarkers IL-6, IL-8, IL-10 and IL-1β to predict donor organs with an excellent outcome (ICU <3 days) as shown in FIG. 4.

### Example 2

Patient Selection: Approximately 32 clinical cases of lung transplantation involving EVLP were identified.

Perfusate Sample: Approximately 1.0 mL of Perfusate solution was removed from the EVLP circuit after 4 hours. Perfusate samples were measured directly or snap frozen and used in the following experiments.

Biomarker Quantification: Perfusate samples were quantified using the Simple Plex System (Protein Simple) using the Ella (Protein Simple) immunoassay platform. Perfusate samples were diluted as per manufacturer's instructions in calibrator diluent. A set of standards for the generation of a standard curve was prepared concurrently (Protein Simple). Each plate was prepared according to the manufacturer's protocol (Protein Simple). Each plate was then run and read on the Simple Plex System (Protein Simple), which was set up and calibrated as per the manufacturer's instructions. Levels of various markers including IL-6, IL-8, IL-10, IL1β, sTNFR1 and sTREM1 were quantified.

Scoring Metric: Simple logistic regression was used to determine the weighting of each biomarker for the decision to transplant following EVLP (accept or decline) (n=32).

Statistical Analysis: Analysis was carried out using Prism 7 (GraphPad), SPSS Statistics (IBM), or R software environment. For all statistical calculations, a p-value of less than 0.05 was considered statistically significant.

### Results

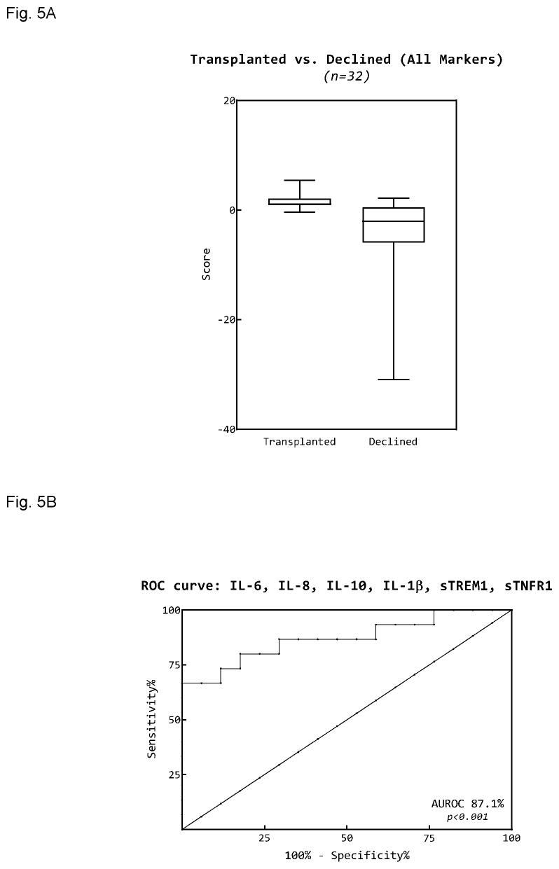

FIG. 5 demonstrates the EVLP Outcome Predictive Performance when IL-6, CXCL8, IL-10, IL-1β, sTNFR1, sTREM1 biomarkers are assessed. FIG. 5A shows a comparison of transplanted and declined lungs when scored using IL-6, CXCL8, IL-10, IL-1β, sTNFR1, sTREM1 as biomarkers during clinical EVLP cases (n=32). FIG. 5B shows the corresponding area under the ROC curve for EVLP outcome prediction.

### Example 3

Additional combinations were assessed using the samples and methods described in Example 2. The Area under the Curve was determined and significance determined.

TABLE 2

| AUROC (%) | p-value | IL-6 | IL-8 | IL-10 | IL-1β |
|---|---|---|---|---|---|
| 83.1 | <0.01 | X | X | X | |
| 83.5 | <0.01 | X | X | | X |

TABLE 3

| AUROC | | | | | | | |
|---|---|---|---|---|---|---|---|
| (%) | p-value | IL-6 | IL-8 | IL-10 | IL-1β | sTREM1 | sTNFR1 |
| 85.9 | <0.001 | X | X | X | | | X |
| 83.9 | <0.01 | X | X | | X | | X |
| 83.5 | <0.01 | X | X | | X | X | |
| 83.5 | <0.01 | | X | X | X | | X |
| 82.8 | <0.01 | X | X | X | | X | |
| 80.4 | <0.01 | | X | X | X | X | |
| 76.5 | <0.05 | X | | X | X | X | |
| 71.4 | <0.05 | X | | X | X | | X |

TABLE 4

| AUROC | | | | | | | |
|---|---|---|---|---|---|---|---|
| (%) | p-value | IL-6 | IL-8 | IL-10 | IL-1β | sTREM1 | sTNFR1 |
| 86.7 | <0.001 | X | X | X | | X | X |
| 87.1 | <0.001 | X | X | | X | X | X |

Other combinations tested included:

IL1β-sTNFR1-sTREM1; AUROC=75.3 p<0.05

IL-10-sTNFR1-sTREM1; AUROC=78.0 p<0.01

In other experiments, it was found that TNFR1 and sTREM1 correlate (0.16 and 0.23, p<0.05, 0.01 respectively) with days on a ventilator.

### Example 4

Biochemical and physiological parameters were correlated with biomarker levels in early (1 h) and late (4) EVLP test perfusates.

Tables 5 and 6 show the correlation coefficients (r) for a plurality of biochemical (Table 5) and physiological (Table 6) parameters versus the biological (e.g. biomarkers) parameters described herein. Two time points (early (e.g. 1 HR) and late (e.g. 4 HR)) are compared during the EVLP procedure. The extent of the positive correlation or negative correlation is shown according to the depth of shading. The p-values are listed beside each correlation coefficient, and each analysis comprised an N between 60 and 343 sample (N=60-343). The features as shown in the next example can be considered in a combination model.

TABLE 5
Relationships between biological and biochemical parameters during EVLP.

| | | Base Excess (BE) | | | | Bicarbonate | | | |
|---|---|---|---|---|---|---|---|---|---|
| | | Early | | Late | | Early | | Late | |
| | | EVLP | p-value | EVLP | p-value | EVLP | p-value | EVLP | p-value |
| Early | IL-8 | −0.327 | 1.62E−07 | −0.388 | 1.88E−09 | −0.267 | 2.40E−05 | −0.359 | 3.33E−08 |
| EVLP | IL-6 | −0.421 | 5.69E−12 | −0.525 | 1.45E−17 | −0.406 | 4.13E−11 | −0.486 | 1.11E−14 |
| | sTREM1 | −0.417 | 1.09E−11 | −0.322 | 8.70E−07 | −0.253 | 6.56E−05 | −0.279 | 2.36E−05 |
| | sTNFR1 | −0.282 | 7.25E−06 | −0.267 | 5.29E−05 | −0.113 | 7.88E−02 | −0.181 | 6.50E−03 |
| Late | sTNFR1 | −0.329 | 1.24E−07 | −0.458 | 4.60E−13 | −0.280 | 8.87E−06 | −0.440 | 4.46E−12 |
| EVLP | IL-1B | −0.327 | 6.89E−03 | −0.426 | 5.60E−04 | −0.193 | 1.18E−01 | −0.282 | 2.63E−02 |
| | sTREM | −0.437 | 7.57E−13 | −0.433 | 1.13E−11 | −0.301 | 1.64E−06 | −0.413 | 1.29E−10 |
| | IL-10 | −0.232 | 5.87E−02 | −0.295 | 1.99E−02 | −0.113 | 3.63E−01 | −0.207 | 1.07E−01 |
| | IL-6 | −0.305 | 1.09E−06 | −0.391 | 1.26E−09 | −0.259 | 4.18E−05 | −0.333 | 3.19E−07 |
| | IL-8 | −0.194 | 2.22E−03 | −0.288 | 1.13E−05 | −0.126 | 4.83E−02 | −0.204 | 2.06E−03 |

pH

| | | Early | | Late | |
|---|---|---|---|---|---|
| | | EVLP | p-value | EVLP | p-value |
| Early | IL-8 | −0.230 | 1.04E−04 | −0.352 | 3.55E−09 |
| EVLP | IL-6 | −0.337 | 7.54E−09 | −0.503 | 9.19E−19 |
| | sTREM1 | −0.391 | 1.19E−11 | −0.339 | 1.45E−08 |
| | sTNFR1 | −0.266 | 6.38E−06 | −0.252 | 3.24E−05 |
| Late | sTNFR1 | −0.292 | 5.63E−07 | −0.421 | 5.19E−13 |
| EVLP | IL-1B | −0.377 | 3.81E−05 | −0.453 | 3.17E−06 |
| | sTREM | −0.436 | 1.69E−14 | −0.437 | 5.89E−14 |
| | IL-10 | −0.266 | 4.46E−03 | −0.413 | 2.60E−05 |
| | IL-6 | −0.189 | 6.38E−04 | −0.345 | 9.86E−10 |
| | IL-8 | −0.112 | 4.55E−02 | −0.229 | 6.65E−05 |

| | | Potassium | | | | Sodium | | | |
|---|---|---|---|---|---|---|---|---|---|
| | | Early | | Late | | Early | | Late | |
| | | EVLP | p-value | EVLP | p-value | EVLP | p-value | EVLP | p-value |
| Early | IL-8 | 0.032 | 6.08E−01 | 0.086 | 1.80E−01 | −0.138 | 3.02E−02 | −0.138 | 3.26E−02 |
| EVLP | IL-6 | 0.063 | 3.16E−01 | 0.159 | 1.28E−02 | −0.100 | 1.15E−01 | −0.138 | 3.32E−02 |
| | sTREM1 | −0.188 | 2.79E−03 | −0.142 | 2.72E−02 | 0.030 | 6.36E−01 | −0.083 | 2.02E−01 |
| | sTNFR1 | −0.144 | 2.17E−02 | −0.107 | 9.68E−02 | 0.137 | 3.09E−02 | −0.054 | 4.02E−01 |
| Late | sTNFR1 | 0.014 | 8.19E−01 | 0.061 | 3.46E−01 | 0.055 | 3.83E−01 | −0.040 | 5.36E−01 |
| EVLP | IL-1B | 0.081 | 5.09E−01 | 0.186 | 1.32E−01 | −0.007 | 9.57E−01 | −0.016 | 8.98E−01 |
| | sTREM | −0.098 | 1.20E−01 | −0.028 | 6.60E−01 | −0.034 | 5.95E−01 | −0.009 | 8.85E−01 |
| | IL-10 | −0.014 | 9.13E−01 | 0.102 | 4.12E−01 | 0.064 | 6.09E−01 | 0.058 | 6.39E−01 |
| | IL-6 | 0.016 | 8.03E−01 | −0.037 | 5.67E−01 | 0.038 | 5.50E−01 | 0.103 | 1.09E−01 |
| | IL-8 | 0.096 | 1.29E−01 | 0.030 | 6.45E−01 | 0.116 | 6.84E−02 | 0.161 | 1.25E−02 |
| | | Calcium | | | | Chloride | | | |
| | | Early | | Late | | Early | | Late | |
| | | EVLP | p-value | EVLP | p-value | EVLP | p-value | EVLP | p-value |
| Early | IL-8 | 0.253 | 5.67E−05 | 0.366 | 9.25E−09 | −0.057 | 3.72E−01 | −0.088 | 1.74E−01 |
| EVLP | IL-6 | 0.233 | 2.26E−04 | 0.335 | 1.80E−07 | −0.045 | 4.84E−01 | −0.068 | 2.95E−01 |
| | sTREM1 | 0.143 | 2.47E−02 | 0.203 | 1.96E−03 | −0.036 | 5.68E−01 | −0.068 | 2.93E−01 |
| | sTNFR1 | 0.184 | 3.72E−03 | 0.298 | 3.90E−06 | 0.004 | 9.48E−01 | −0.016 | 8.04E−01 |
| Late | sTNFR1 | 0.064 | 3.20E−01 | 0.327 | 3.56E−07 | 0.002 | 9.71E−01 | 0.038 | 5.50E−01 |
| EVLP | IL-1B | 0.144 | 2.41E−01 | 0.398 | 8.57E−04 | 0.159 | 1.96E−01 | 0.191 | 1.21E−01 |
| | sTREM | 0.143 | 2.54E−02 | 0.268 | 3.80E−05 | −0.030 | 6.39E−01 | 0.011 | 8.70E−01 |
| | IL-10 | 0.134 | 2.74E−01 | 0.354 | 3.32E−03 | 0.216 | 7.63E−02 | 0.181 | 1.42E−01 |
| | IL-6 | −0.043 | 4.98E−01 | 0.243 | 1.85E−04 | 0.040 | 5.25E−01 | 0.101 | 1.18E−01 |
| | IL-8 | −0.058 | 3.61E−01 | 0.272 | 2.64E−05 | 0.017 | 7.86E−01 | 0.129 | 4.48E−02 |
| | | Glucose | | | | Lactate | | | |
| | | Early | | Late | | Early | | Late | |
| | | EVLP | p-value | EVLP | p-value | EVLP | p-value | EVLP | p-value |
| Early | IL-8 | −0.293 | 5.40E−07 | −0.381 | 5.91E−11 | 0.340 | 5.19E−06 | 0.399 | 7.86E−08 |
| EVLP | IL-6 | −0.451 | 1.73E−15 | −0.500 | 3.54E−19 | 0.392 | 1.06E−07 | 0.436 | 3.04E−09 |
| | sTREM1 | −0.385 | 2.21E−11 | −0.376 | 1.15E−10 | 0.342 | 4.56E−06 | 0.358 | 1.88E−06 |
| | sTNFR1 | −0.195 | 9.90E−04 | −0.214 | 3.39E−04 | 0.263 | 5.01E−04 | 0.281 | 2.16E−04 |
| Late | sTNFR1 | −0.324 | 2.20E−08 | −0.460 | 4.77E−16 | 0.415 | 1.20E−08 | 0.448 | 8.18E−10 |
| EVLP | IL-1B | −0.250 | 7.25E−03 | −0.295 | 2.94E−03 | 0.230 | 3.22E−02 | 0.293 | 1.03E−02 |
| | sTREM | −0.427 | 4.95E−14 | −0.509 | 5.23E−20 | 0.404 | 3.35E−08 | 0.462 | 1.95E−10 |
| | IL-10 | −0.243 | 9.05E−03 | −0.303 | 2.19E−03 | 0.234 | 2.89E−02 | 0.304 | 7.55E−03 |
| | IL-6 | −0.221 | 5.90E−05 | −0.294 | 1.54E−07 | 0.189 | 5.68E−03 | 0.242 | 5.33E−04 |
| | IL-8 | −0.059 | 2.91E−01 | −0.119 | 3.61E−02 | 0.089 | 1.96E−01 | 0.102 | 1.50E−01 |

TABLE 6
Relationships between biological and physiological parameters during EVLP

| | | Peak Pressure | | | | PA Pressure | | | |
|---|---|---|---|---|---|---|---|---|---|
| | | Early | | Late | | Early | | Late | |
| | | EVLP | p-value | EVLP | p-value | EVLP | p-value | EVLP | p-value |
| Early | IL-8 | 0.160 | 1.20E−02 | 0.164 | 9.70E−03 | 0.097 | 9.83E−02 | 0.094 | 1.11E−01 |
| EVLP | IL-6 | 0.073 | 2.55E−01 | 0.074 | 2.42E−01 | 0.085 | 1.48E−01 | 0.085 | 1.48E−01 |
| | sTREM1 | 0.169 | 7.90E−03 | 0.062 | 3.31E−01 | 0.123 | 3.55E−02 | 0.100 | 8.81E−02 |
| | sTNFR1 | 0.093 | 1.45E−01 | 0.061 | 3.34E−01 | 0.006 | 9.21E−01 | 0.114 | 5.21E−02 |
| Late | sTNFR1 | 0.218 | 5.36E−04 | 0.189 | 2.77E−03 | −0.050 | 3.87E−01 | −0.012 | 8.31E−01 |
| EVLP | IL-1B | 0.166 | 1.80E−01 | 0.339 | 5.32E−03 | −0.090 | 3.40E−01 | 0.014 | 8.91E−01 |
| | sTREM1 | 0.257 | 4.44E−05 | 0.126 | 4.73E−02 | 0.135 | 2.08E−02 | 0.092 | 1.16E−01 |
| | IL-10 | 0.253 | 3.89E−02 | 0.284 | 2.09E−02 | −0.112 | 2.36E−01 | 0.088 | 3.82E−01 |
| | IL-6 | 0.191 | 2.53E−03 | 0.261 | 2.93E−05 | −0.013 | 8.11E−01 | 0.028 | 6.13E−01 |
| | IL-8 | 0.157 | 1.35E−02 | 0.304 | 9.37E−07 | −0.034 | 5.33E−01 | −0.002 | 9.72E−01 |
| | | LA Pressure | | | | Driving Pressure | | | |
| | | Early | | Late | | Early | | Late | |
| | | EVLP | p-value | EVLP | p-value | EVLP | p-value | EVLP | p-value |
| Early | IL-8 | 0.011 | 8.45E−01 | −0.037 | 5.25E−01 | 0.187 | 3.05E−03 | 0.183 | 4.37E−03 |
| EVLP | IL-6 | −0.006 | 9.24E−01 | −0.051 | 3.83E−01 | 0.071 | 2.64E−01 | 0.037 | 5.67E−01 |
| | sTREM1 | −0.043 | 4.63E−01 | −0.081 | 1.71E−01 | 0.114 | 7.40E−02 | −0.035 | 5.89E−01 |
| | sTNFR1 | 0.009 | 8.72E−01 | −0.051 | 3.88E−01 | 0.137 | 3.12E−02 | 0.083 | 2.00E−01 |
| Late | sTNFR1 | −0.070 | 2.33E−01 | −0.116 | 4.74E−02 | 0.165 | 9.24E−03 | 0.135 | 3.60E−02 |
| EVLP | IL-1B | 0.001 | 9.90E−01 | 0.027 | 7.87E−01 | 0.149 | 2.37E−01 | 0.247 | 4.77E−02 |
| | sTREM1 | −0.069 | 2.41E−01 | −0.114 | 5.15E−02 | 0.156 | 1.38E−02 | 0.019 | 7.70E−01 |
| | IL-10 | 0.000 | 1.00 | 0.066 | 5.12E−01 | 0.273 | 2.79E−02 | 0.224 | 7.25E−02 |
| | IL-6 | 0.089 | 1.02E−01 | 0.024 | 6.61E−01 | 0.095 | 1.31E−01 | 0.136 | 3.43E−02 |
| | IL-8 | 0.119 | 2.89E−02 | 0.052 | 2.64E−01 | 0.078 | 2.18E−01 | 0.238 | 1.90E−04 |
| | | Dynamic Compliance | | | | Static Compliance | | | |
| | | Early | | Late | | Early | | Late | |
| | | EVLP | p-value | EVLP | p-value | EVLP | p-value | EVLP | p-value |
| Early | IL-8 | −0.159 | 1.25E−02 | −0.168 | 8.57E−03 | −0.184 | 3.73E−03 | −0.240 | 1.45E−04 |
| EVLP | IL-6 | −0.045 | 4.86E−01 | −0.023 | 7.21E−01 | −0.019 | 7.63E−01 | −0.022 | 7.36E−01 |
| | sTREM1 | −0.048 | 4.53E−01 | 0.139 | 3.03E−02 | 0.112 | 8.02E−02 | 0.168 | 8.57E−03 |
| | sTNFR1 | −0.015 | 8.17E−01 | −0.036 | 5.76E−01 | −0.056 | 3.81E−01 | −0.080 | 2.11E−01 |
| Late | sTNFR1 | −0.164 | 9.70E−03 | −0.148 | 2.06E−02 | −0.111 | 8.18E−02 | −0.124 | 5.25E−02 |
| EVLP | IL-1B | −0.217 | 8.03E−02 | −0.407 | 8.50E−04 | −0.172 | 1.63E−01 | −0.301 | 1.41E−02 |
| | sTREM1 | −0.191 | 2.61E−03 | 0.012 | 8.58E−01 | 0.011 | 8.66E−01 | 0.093 | 1.46E−01 |
| | IL-10 | −0.204 | 1.00E−01 | −0.311 | 1.24E−02 | −0.226 | 6.57E−02 | −0.278 | 2.38E−02 |
| | IL-6 | −0.007 | 9.08E−01 | −0.055 | 3.91E−01 | 0.093 | 1.44E−01 | −0.013 | 8.40E−01 |
| | IL-8 | −0.110 | 8.38E−02 | −0.236 | 1.95E−04 | −0.075 | 2.39E−01 | −0.235 | 2.06E−04 |

PVR

| | | Early | | Late | |
|---|---|---|---|---|---|
| | | EVLP | p-value | EVLP | p-value |
| Early | IL-8 | 0.133 | 5.46E−02 | 0.175 | 1.04E−02 |
| EVLP | IL-6 | 0.076 | 2.75E−01 | 0.061 | 3.80E−01 |
| | sTREM1 | 0.013 | 8.51E−01 | −0.023 | 7.35E−01 |
| | sTNFR1 | 0.009 | 8.91E−01 | 0.147 | 3.25E−02 |
| Late | sTNFR1 | −0.204 | 2.86E−03 | −0.129 | 5.89E−02 |
| EVLP | IL-1B | −0.032 | 7.44E−01 | 0.040 | 7.02E−01 |
| | sTREM1 | −0.017 | 8.11E−01 | −0.102 | 1.38E−01 |
| | IL-10 | −0.086 | 3.78E−01 | 0.070 | 5.01E−01 |
| | IL-6 | −0.067 | 2.94E−01 | 0.016 | 8.09E−01 |
| | IL-8 | −0.007 | 9.15E−01 | 0.145 | 2.37E−02 |

| | | PO₂ | | | | PCO₂ | | | |
|---|---|---|---|---|---|---|---|---|---|
| | | Early | | Late | | Early | | Late | |
| | | EVLP | p-value | EVLP | p-value | EVLP | p-value | EVLP | p-value |
| Early | IL-8 | −0.046 | 4.33E−01 | −0.086 | 1.44E−01 | 0.092 | 1.80E−01 | 0.253 | 3.23E−04 |
| EVLP | IL-6 | −0.082 | 1.66E−01 | −0.148 | 1.20E−02 | 0.107 | 1.18E−01 | 0.275 | 8.16E−05 |
| | sTREM1 | −0.135 | 2.19E−02 | −0.078 | 1.88E−01 | −0.027 | 6.93E−01 | 0.029 | 5.83E−01 |
| | sTNFR1 | −0.083 | 1.57E−01 | −0.131 | 2.64E−02 | 0.156 | 2.31E−02 | 0.283 | 5.18E−05 |
| Late | sTNFR1 | −0.129 | 2.71E−02 | −0.173 | 3.09E−03 | −0.058 | 4.01E−01 | 0.139 | 5.02E−02 |
| EVLP | IL-1B | −0.246 | 8.73E−03 | −0.439 | 4.28E−06 | 0.212 | 8.71E−02 | 0.191 | 1.44E−01 |
| | sTREM1 | −0.186 | 1.43E−03 | −0.137 | 1.93E−02 | −0.079 | 2.54E−01 | 0.022 | 7.57E−01 |
| | IL-10 | −0.247 | 8.39E−03 | −0.467 | 8.77E−07 | 0.178 | 1.53E−01 | 0.075 | 5.71E−01 |
| | IL-6 | −0.169 | 2.00E−03 | −0.297 | 5.64E−08 | −0.093 | 1.77E−01 | −0.030 | 6.79E−01 |
| | IL-8 | −0.098 | 7.51E−02 | −0.277 | 4.65E−07 | −0.014 | 8.36E−01 | 0.021 | 7.72E−01 |

### Example 5

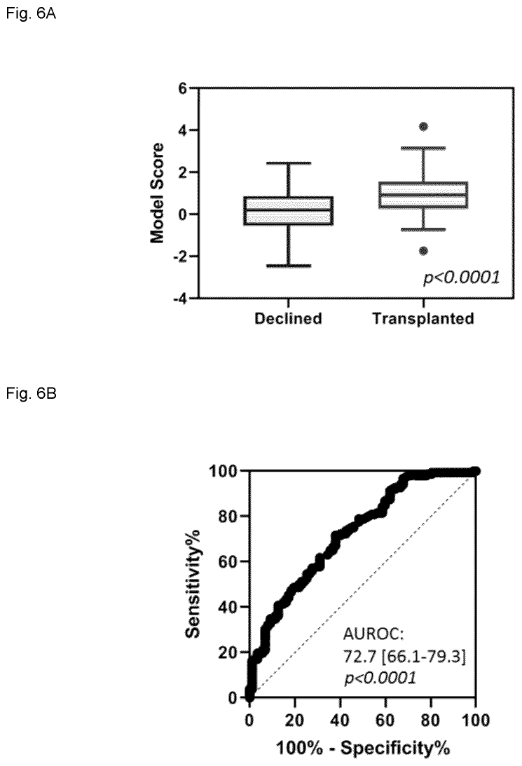

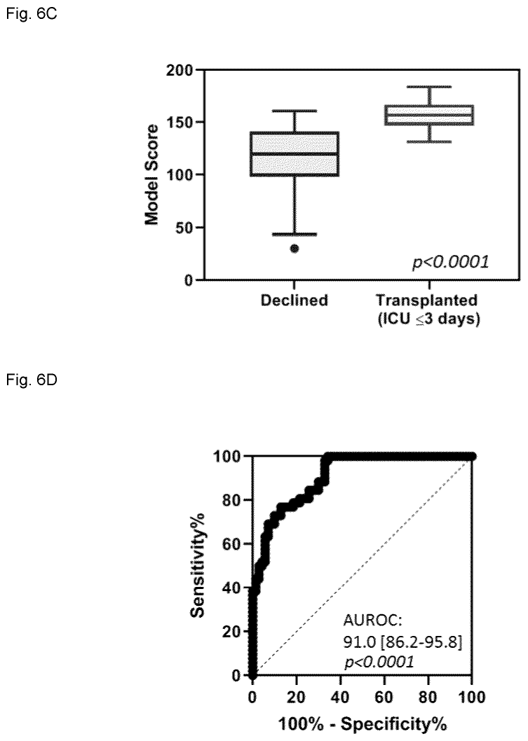

FIG. 6 demonstrates that the combined model including biochemical and/or physiologic data in combination with biomarker data provides for a robust assessment tool.

Panels A-B show a biological+biochemical combination of pH, IL-6 & IL-8, sTNFR1, and sTREM1 predict a suitable organ for transplantation

Panels C-D show biological+physiological combination of PO2, IL-6 & IL-8, and sTNFR1 to predict a lung likely to result in an excellent outcome (ICU ≤3 days).

The data were generated using stepwise logistic regression.

For Panel A-B, risk is calculated according the formula:

Risk=a₀+a₁(pH)+a₂(IL-6&IL-8)+a₃(sTNFR1)+a₄(sTREM1)
Where a₀₋₄ are coefficients.

For Panel C-D risk is calculated according the formula:

Risk=b₀+b₁(PO₂)+b₂(IL-6&IL-8)+b₃(sTNFR1)
Where b₀₋₃ are coefficients.

A base model was built using the physiological/biochemical parameters. The biomarker data was added to the model to assess for significant improvement.

In the present examples, the biochemical or physiological data was used to predict suitability and outcome and then combined with the biomarker data (FIG. 6) (N=173). As demonstrated, the addition of biological data provides a significant improvement to either biochemical and/or physiological assessments alone. Similarly it was determined that the reverse is also true e.g. the physiological data improves predictions based on the biological.

The combination model performance, as measured by the area under the ROC curve, is shown in FIG. 6 (N=173) and shows that an improvement is obtained when combining biochemical and physiological parameters with biomarker levels. p-values are indicated in the top left corner of each graph.

The reverse was also assessed. A base model was built using biomarker parameter values assessed in Panels C and D. Physiological parameter values were then added to the model to assess for significant improvement. For these variables the significance and performance (AUROC) of when physiological parameter values were added to biomarker parameters is the same as when the biomarker parameters are added to physiological parameters.

### Example 6

Other combinations of parameters and biomarker data are assessed. Different groupings of the factors are assessed for different predictions, including for which best predict ICU LOS or Hospital LOS or APACHE II score, etc.

### CITATIONS FOR REFERENCES REFERRED TO IN THE SPECIFICATION

1. M. Cypel, S. Keshavjee, The clinical potential of ex vivo lung perfusion. Expert Rev Respir Med 6, 27-35 (2012).
2. M. Cypel, M. Rubacha, J. Yeung, S. Hirayama, K. Torbicki, M. Madonik, S. Fischer, D. Hwang, A. Pierre, T. K. Waddell, M. de Perrot, M. Liu, S. Keshavjee, Normothermic ex vivo perfusion prevents lung injury compared to extended cold preservation for transplantation. Am J Transplant 9, 2262-2269 (2009).
3. M. Cypel, J. C. Yeung, S. Hirayama, M. Rubacha, S. Fischer, M. Anraku, M. Sato, S. Harwood, A. Pierre, T. K. Waddell, M. de Perrot, M. Liu, S. Keshavjee, Technique for prolonged normothermic ex vivo lung perfusion. J Heart Lung Transplant 27, 1319-1325 (2008).
4. M. Cypel, J. C. Yeung, M. Liu, M. Anraku, F. Chen, W. Karolak, M. Sato, J. Laratta, S. Azad, M. Madonik, C. W. Chow, C. Chaparro, M. Hutcheon, L. G. Singer, A. S. Slutsky, K. Yasufuku, M. de Perrot, A. F. Pierre, T. K. Waddell, S. Keshavjee, Normothermic ex vivo lung perfusion in clinical lung transplantation. N Engl J Med 364, 1431-1440 (2011).
5. J. C. Yeung, M. Cypel, E. Massad, S. Keshavjee, Ex vivo lung perfusion and reconditioning. Multimed Man Cardiothorac Surg 2011, mmcts 2009 004242 (2011).
6. M. Cypel, J. C. Yeung, S. Keshavjee, Novel approaches to expanding the lung donor pool: donation after cardiac death and ex vivo conditioning. Clin Chest Med 32, 233-244 (2011).
7. M. K. Hsin, I. Iskender, D. Nakajima, M. Chen, H. Kim, P. R. dos Santos, J. Sakamoto, J. Lee, K. Hashimoto, C. Harmantas, D. Hwang, T. Waddell, M. Liu, S. Keshavjee, M. Cypel, Extension of donor lung preservation with hypothermic storage after normothermic ex vivo lung perfusion. J Heart Lung Transplant 35, 130-136 (2016).
8. T. N. Machuca, O. Mercier, S. Collaud, J. Tikkanen, T. Krueger, J. C. Yeung, M. Chen, S. Azad, L. Singer, K. Yasufuku, M. de Perrot, A. Pierre, T. K. Waddell, S. Keshavjee, M. Cypel, Lung transplantation with donation after circulatory determination of death donors and the impact of ex vivo lung perfusion. Am J Transplant 15, 993-1002 (2015).
9. T. N. Machuca, M. Cypel, J. C. Yeung, R. Bonato, R. Zamel, M. Chen, S. Azad, M. K. Hsin, T. Saito, Z. Guan, T. K. Waddell, M. Liu, S. Keshavjee, Protein expression profiling predicts graft performance in clinical ex vivo lung perfusion. Ann Surg 261, 591-597 (2015).

1. A method for the screening, diagnosing, or detecting of outcome/risk as it relates to donor lungs, comprising:
determining, in one or more test EVLP perfusate samples of a donor lung, one or more parameter values related to a level of polypeptide of at least three biomarkers selected from IL-6, IL-8, IL-10, and IL-1β, optionally in combination with one or both of sTNFR1 and sTREM1;
optionally determining in one or more test EVLP perfusate samples of the donor lung, one or more biochemical parameter values and/or determining one or more physiological parameter values of the donor lung, the biochemical data optionally comprising at least one of: base excess, bicarbonate, potassium, sodium, calcium, chloride, glucose, lactate and pH, and the physiological data optionally comprising at least one of: driving pressure, PCO₂ (measured and differential), PO₂ (measured and differential), airway pressure, static and dynamic compliance, PA and/or LA pressure, and pulmonary vascular resistance,
and
comparing the one or more parameter values related to a level of each the at least three biomarkers in the one or more perfusate samples with control EVLP data or a cut-off level, wherein the differential level is indicative of outcome/risk optionally after transplant; and/or
using the one or more parameter values related to a level of the at least three biomarkers in combination, and optionally the more or more biochemical and/or physiological parameter value(s) as part of an algebraic calculation or model of outcome/risk optionally after transplant,
optionally wherein the outcome/risk is risk of a negative post-lung transplant patient outcome (PO) and/or determined to be sufficient to discontinue EVLP, and
optionally wherein the PO is selected from extended intensive care unit (ICU) length of stay, extended time on ventilator and/or extended post-transplant hospital stay.

2.-4. (canceled)

5. The method of claim 1 for predicting a patient outcome (PO) variable for a lung transplant recipient after transplant, wherein the determining step comprises:
obtaining one or more test EVLP perfusate samples of a perfusion solution collected during perfusion of a donor lung;
determining in the one or more test EVLP perfusate samples one or more parameter values related to a polypeptide level of at least three biomarkers selected from IL-6, IL-8, IL-10, and IL-1β, and optionally one or both of sTNFR1 and sTREM1;
optionally determining in one or more test EVLP perfusate samples of the donor lung, one or more biochemical parameter values and/or determining one or more physiological parameter values of the donor lung;
optionally generating a PO variable score for the donor lung based on the one or more parameter values; and
comparing one or more parameter values or optionally the PO score for the donor lung with control EVLP data or a cut-off level, wherein the PO variable score is indicative of a PO variable after transplant,
optionally wherein the outcome/risk or PO variable is selected from ICU length of stay, post-transplant hospital length of stay, number of days on a ventilator, APACHE score and post graft dysfunction (PGD) grade, optionally PGD0/1 or PGD3.

6.-11. (canceled)

12. The method of claim 1, further comprising selecting said donor lung for transplant if the outcome/risk is acceptable or below the cut-off value, preparing said donor lung for transplant and/or transplanting said donor lung into a suitable recipient, or discarding the donor lung and/or using the donor lung for research or other purposes if the outcome/risk or is unacceptable or above the cut-off value.

13. (canceled)

14. The method of claim 1, for the early detection of donor lungs that will be declined at the end of the EVLP process comprising:
obtaining one or more test EVLP perfusate samples of a perfusion EVLP solution collected during perfusion of the donor lung;
determining in the one or more test EVLP perfusate samples one or more parameter values related to a level of at least three biomarkers selected from IL-6, IL-8, IL-10, and IL-1β, and optionally one or both of sTNFR1 and sTREM1;
optionally determining in one or more test EVLP perfusate samples of the donor lung, one or more biochemical parameter values and/or determining one or more physiological parameter values of the donor lung; and
optionally generating a transplant suitability score for the donor lung based on the one or more parameter values;
comparing the one or more parameter values or optionally the transplant suitability score for the donor lung with control EVLP data or a cut-off level, and
continuing perfusion if the one or more parameter values or transplant suitability score indicates that the donor lung is suitable or may be suitable for transplantation and discontinuing perfusion if the one or more parameter values or the transplant suitability score indicates that the donor lung will be declined for transplantation, and
optionally discarding the donor lung and/or using the donor lung for research or other purposes if one or more parameter values or transplant suitability score indicates that the lung will be declined for transplantation after EVLP.

15. (canceled)

16. The method of claim 1 for selecting a candidate donor lung for transplant, the method comprising:
obtaining one or more test EVLP perfusate samples of a perfusion solution collected during perfusion of the donor lung;
determining one or more parameter values related to a level of at least three biomarkers selected from IL-6, IL-8, IL-10, and IL-1β and optionally one or both of sTNFR1 and sTREM1, in the one or more test EVLP perfusate samples;
a) optionally generating a transplant suitability score for the donor lung based on the one or more parameter values;
b) comparing the one or more parameter values or optionally the transplant suitability score for the donor lung with control EVLP data or a cut-off level; and
selecting the donor lung for transplant according to the one or more parameter values or the transplant suitability score.

17. The method of claim 1, wherein the method first comprises:
inserting the donor lung into a perfusion machine;
using the perfusion machine to perfuse the donor lung with an EVLP solution;
wherein the one or more test EVLP perfusate samples are obtained from the EVLP solution during perfusion of the donor lung.

18. The method of claim 1, wherein the one or more test EVLP perfusate samples are collected after at least or at about 45 min of EVLP, after at least or at about 1 hour of EVLP, after at least or at about 75 min of EVLP, after at least or at about 1.5 hours of EVLP, after at least or at about 2 hours of EVLP, after at least or at about 2.5 hours of EVLP, after at least or at about 3 hours of EVLP, after at least or at about 3.5 hours of EVLP and/or after at least or at about 4 hours of EVLP, preferably after at least or at about 45 min and/or about 4 hours, or any time therebetween, optionally between 1 hour and 4 hours of EVLP, between 1 hour and 3 hours of EVLP, between 1.5 hour and 3 hours of EVLP, between 1.5 hours and 2.5 hours of EVLP or between 1 hour and 2 hours of EVLP.

19. The method of claim 1, wherein a first test EVLP perfusate sample is collected after at least or at about 45 min of EVLP, and one or more subsequent test EVLP perfusate samples are collected any time therebetween 1 min and 6 hours of collecting the first perfusate sample, optionally intervening time between collecting any two test EVLP perfusate samples is about 1, 2, 3, 4, 5, 6, 7, 8, 9, 10, 11, 12, 15, 20, 25, 30, 35, 40, 45, 50, 55, 60, 65, 70, 75, 80, 85, 90, 100, 105, 110, 120, 135, 150, 165, 180 min, optionally about 2, 2.5, 3, 3.5, 4, 4.5, 5, 5.5 or 6 hours, optionally any time therebetween 1 min and 6 hours.

20. The method of claim 1, wherein the one or more parameter values comprises a concentration of the at least three biomarkers, a rate of biomarker production of the at least three biomarkers and/or a ratio of the concentration of each of the at least three biomarkers, optionally wherein the ratio is the concentration of a subsequent perfusate sample/concentration of an earlier perfusate sample.

21.-22. (canceled)

23. The method of claim 1, wherein the at least three biomarkers measured comprise or are:
IL-8, IL-6, and IL-10 and optionally one or both of sTNFR1 and sTREM1;
IL-6, IL-8, and IL-1β, and optionally one or both of sTNFR1 and sTREM1;
IL-6, IL-10, and IL-1β and optionally one or both of sTNFR1 and sTREM1;
IL-8, IL-10 and IL-1β and optionally one or both of sTNFR1 and sTREM1; or
IL-6, IL-8, IL-10 and IL-1β and optionally one or both of sTNFR1 and sTREM1.

24.-27. (canceled)

28. The method of claim 5, wherein the one or more parameter values comprises:
rate of IL-8 production and optionally rate of one or both of sTNFR1 and sTREM1 production;
rate of IL-6 production, and optionally rate of one or both of sTNFR1 and sTREM1 production;
rate of IL-10 production, and optionally rate of one or both of sTNFR1 and sTREM1 production;
rate of IL-1β production, and optionally rate of one or both of sTNFR1 and sTREM1 production;
concentration of IL-8, and optionally concentration of one or both of sTNFR1 and sTREM1, wherein the one or more perfusate samples is a perfusate sample taken after at least or at about 45 min of perfusion, optionally a perfusate sample taken after about 4 hours of perfusion;
concentration of IL-6 and optionally concentration of one or both of sTNFR1 and sTREM1, wherein the one or more perfusate samples is a perfusate sample taken after at least or at about 45 min of perfusion, optionally a perfusate sample taken after about 4 hours of perfusion;
concentration of IL-10, and optionally concentration of one or both of sTNFR1 and sTREM1 wherein the one or more perfusate samples is a perfusate sample taken after at least or at about 45 min of perfusion, optionally a perfusate sample taken after about 4 hours of perfusion; and/or
concentration of IL-1β and optionally concentration of one or both of sTNFR1 and sTREM1, wherein the one or more perfusate samples is a perfusate sample taken after at least or at about 45 min of perfusion, optionally a perfusate sample taken after about 4 hours of perfusion.

29.-39. (canceled)

40. The method of claim 1, wherein the one or more parameter values are selected from: 1) IL-6 concentration and optionally concentration of one or both of sTNFR1 and sTREM1 after at least or at about 1 hour of perfusion and about 4 hours of perfusion or any time therebetween, optionally between 1 hour and 3 hours or between 1.5 hours and 2.5 hours of perfusion; 2) IL-8 concentration and optionally concentration of one or both of sTNFR1 and sTREM1 at about 2 hours, about 3 hours or about 4 hours of perfusion; 3) IL-10 concentration and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion; 4) IL-1β concentration and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 5) IL-6 and IL-8 concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 6) IL-6 and IL-10 concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 7) IL-6 and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 8) IL-8 and IL-10 concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 9) IL-8 and IL-1β and optionally concentration of one or both of sTNFR1 and sTREM1 concentrations after at least or at about 1 hour of perfusion and about 4 hours of perfusion optionally between 1 hour and 3 hours or between 1.5 hours and 2.5 hours of perfusion, 10) IL-10 and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 11) IL-6, IL-8 and IL-10 concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 12) IL-6, IL-8, and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 13) IL-6, IL-10, and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, 14) IL-8, IL-10, and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion, and 15) IL-6, IL-8, IL-10 and IL-1β concentrations and optionally concentration of one or both of sTNFR1 and sTREM1 after at least or at about 1 hour of perfusion and about 4 hours of perfusion optionally between 1 hour and 3 hours or between 1.5 hours and 2.5 hours of perfusion.

41. The method of claim 5, wherein the one or more parameter values comprises a concentration of the at least three biomarkers normalized to total lung capacity (TLC).

42. The method of claim 1, wherein the one or more parameter values related to a level of the at least three biomarkers is detected using an immunoassay such as ELISA and/or, where more than one level of the at least three biomarkers is detected with a multiplex assay.

43. The method of claim 1, wherein the method further comprises determining one or more biochemical parameter values in the one or more test EVLP perfusate samples and/or determining one or more physiological parameter values of the donor lung, comparing the measured parameter value(s) with control EVLP data or a cut-off level, and/or wherein the measured parameter value(s) including the one or more parameter values related to a level of the at least three biomarkers in combination, is used as part of an algebraic calculation or model of outcome/risk, optionally after transplant.

44.-45. (canceled)

46. A kit comprising at least one detection antibody specific for a biomarker selected from IL-8, IL-6, IL-10 and IL-1β, optionally in combination with one or both of sTNFR1 and sTREM1, optionally wherein the detection antibody is coupled to beads or labelled, the kit optionally further comprising one or more of a 96-well plate, standards, assay buffer, wash buffer, sample diluent, standard diluent, detection antibody diluent, streptavidin-PE, a filter plate and sealing tape, optionally for performing the method of claim 1.

47. A computer-implemented method for the detecting of outcome/risk as it relates to donor lungs the method comprising:
obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
querying, via one or more computers, a database of control EVLP data or a cut-off level associated with patient outcome or transplant suitability to compare the EVLP data to the control EVLP data or cut-off level; and
determining a ranking score based on the comparison between the EVLP data and the control EVLP data or cut-off level and
optionally comprising one or more of:
building a base model using biochemical data and/or the physiological data to determine the ranking score, the biochemical data optionally comprising at least one of: base excess, bicarbonate, potassium, sodium, calcium, chloride, glucose, lactate and pH, and the physiological data optionally comprising at least one of: driving pressure, PCO₂ (measured and differential), PO₂ (measured and differential), airway pressure, static and dynamic compliance, PA and/or LA pressure, and pulmonary vascular resistance;
combining the EVLP data to biochemical data and/or the physiological data prior to determining the ranking score;
analyzing the combined data to extract multiple features indicating characteristics of the donor lung or a after transplant risk/outcome, the extracting the multiple features optionally carried out using an artificial intelligence technique or a machine learning technique, optionally wherein the machine learning technique comprises a neural network; and
generating multiple classifiers/scores based on the multiple features, wherein each of the multiple classifiers/scores indicates one of multiple outcomes, the generating multiple classifiers/scores optionally comprising generating the multiple classifiers using the artificial intelligence technique or the machine learning technique.

48.-55. (canceled)

56. A system for the detecting of outcome/risk as it relates to donor lungs, the system comprising:
a first component that is configured to obtain EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
a second component that is configured to query a database of control EVLP data or a cut-off level associated with patient outcome or transplant suitability to compare the EVLP data to the control EVLP data or cut-off level; and
a third component that is configured to determine a ranking score based on the comparison between the EVLP data and control EVLP data or cut-off level, and
optionally comprising one or more of:
a fourth component that is configured to building a base model using biochemical data and/or the physiological data to determine the ranking score or combine the EVLP data to biochemical data and/or the physiological data prior to determining the ranking score, the biochemical data optionally comprising at least one of: base excess, bicarbonate, potassium, sodium, calcium, chloride, glucose, lactate and pH, and the physiological data optionally comprising at least one of: driving pressure, PCO₂ (measured and differential), PO₂ (measured and differential), airway pressure, static and dynamic compliance, PA and/or LA pressure, and pulmonary vascular resistance;
a fifth component that is configured to analyze the combined data to extract multiple features indicating characteristics of the donor lung, the extracting the multiple features optionally carried out using an artificial intelligence technique or a machine learning technique optionally wherein the machine learning technique comprises a neural network; and
a sixth component that is configured to generate multiple classifiers/scores based on the multiple features, wherein each of the multiple classifiers/scores indicates one of multiple outcomes, the generating multiple classifiers/scores optionally comprising generating the multiple classifiers using the artificial intelligence technique or the machine learning technique.

57.-62. (canceled)

63. A non-transitory computer-readable storage medium storing computer-readable instructions for performing the computer implemented method of claim 47, comprising:
instructions for obtaining the EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
instructions for querying the database of control EVLP data or a cut-off level associated with patient outcome or transplant suitability to compare the EVLP data to the control EVLP data or cut-off level; and
instructions for determining the ranking score based on the comparison between the EVLP data and the control EVLP data or cut-off level, and
optionally comprising one or more of:
instructions for building the base model using biochemical data and/or the physiological data to determine the ranking score or combining the EVLP data to biochemical data and/or the physiological data prior to determining the ranking score;
instructions for analyzing the combined data to extract multiple features indicating characteristics of the donor lung;
instructions for generating the multiple classifiers/scores based on the multiple features, wherein each of the multiple classifiers/scores indicates one of multiple outcomes;
instructions for extracting the multiple features using an artificial intelligence technique or a machine learning technique; and
instructions for generating the multiple classifiers using the artificial intelligence technique or the machine learning technique.

64.-66. (canceled)

67. A computer-program product for use in conjunction with an electronic device, the computer-program product comprising the non-transitory computer-readable storage medium of claim 63 and a computer-program mechanism embedded therein, the computer-program mechanism comprising:
the instructions for obtaining EVLP data relating to one or more test EVLP perfusate samples of a donor lung, the EVLP data comprising one or more parameter values relating to a level of polypeptide of at least three biomarkers selected from IL-6, IL-8, IL-10, and IL-1β, optionally in combination with one or both of sTNFR1 and sTREM1;
the instructions for querying a database of control EVLP data or a cut-off level associated with patient outcome or transplant suitability to compare the EVLP data to the control EVLP data or cut-off level; and
the instructions for determining a ranking score based on the comparison between the EVLP data and the control EVLP data or cut-off level.

68.-70. (canceled)
